(19) United States
(12) Patent Application Publication
(10) Pub. No.: US 2006/0078947 A1
(43) Pub. Date: Apr. 13, 2006

(54) Methods and compositions for treating hematological disorders using 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014

(75) Inventors: Louise M. Kelly, Brookline, MA (US); Joseph M. Carroll, Cambridge, MA (US); Deborah Farlow, Waltham, MA (US); Aileen Healy, Medford, MA (US)

Correspondence Address: MILLENNIUM PHARMACEUTICALS, INC., 40 Landsdowne Street, CAMBRIDGE, MA, 02139, US

(73) Assignee: Millennium Pharmaceuticals, Inc.

(21) Appl. No.: 11/288,493

(22) Filed: Nov. 29, 2005

Related U.S. Application Data
(63) Continuation of application No. 10/772,636, filed on Feb. 5, 2004.
(60) Provisional application No. 60/445,241, filed on Feb. 5, 2003.
(60) Provisional application No. 60/448,389, filed on Feb. 18, 2003.
(60) Provisional application No. 60/456,320, filed on Mar. 20, 2003.
(60) Provisional application No. 60/460,279, filed on Apr. 3, 2003.
(60) Provisional application No. 60/465,924, filed on Apr. 28, 2003.
(60) Provisional application No. 60/470,052, filed on May 13, 2003.
(60) Provisional application No. 60/498,106, filed on Aug. 26, 2003.
(60) Provisional application No. 60/500,179, filed on Sep. 4, 2003.
(60) Provisional application No. 60/502,909, filed on Sep. 15, 2003.
(60) Provisional application No. 60/510,351, filed on Oct. 10, 2003.
(60) Provisional application No. 60/512,380, filed on Oct. 17, 2003.

(51) Int. Cl. G01N 33/567; G01N 33/53

(52) U.S. Cl. 435/7.2

(57) ABSTRACT

The present invention relates to methods for the diagnosis and treatment of hematological disorders. Specifically, the present invention identifies the differential expression of 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 and 5014 genes in tissues relating to hematological disorders sensation, relative to their expression in normal, or non-hematological disorders disease states, and/or in response to manipulations relevant to hematological disorders. The present invention describes methods for the diagnostic evaluation and prognosis of various hematological disorders, and for the identification of subjects exhibiting a predisposition to such conditions. The invention also provides methods for identifying a compound capable of modulating hematological disorders. The present invention also provides methods for the identification and therapeutic use of compounds as treatments of hematological disorders.

### RELATED APPLICATIONS

The present application claims the benefit of U.S. Provisional Application Ser. No. 60/445,241, filed on Feb. 5, 2003, of U.S. Provisional Application Ser. No. 60/448,389, filed on Feb. 18, 2003, of U.S. Provisional Application Ser. No. 60/456,320, filed on Mar. 20, 2003, of U.S. Provisional Application Ser. No. 60/460,279, filed on Apr. 3, 2003, of U.S. Provisional Application Ser. No. 60/465,924, filed on Apr. 28, 2003, of U.S. Provisional Application Ser. No. 60/470,052, filed on May 13, 2003, of U.S. Provisional Application Ser. No. 60/498,106, filed on Aug. 26, 2003, of U.S. Provisional Application Ser. No. 60/500,179, filed on Sep. 4, 2003, of U.S. Provisional Application Ser. No. 60/502,909, filed on Sep. 15, 2003, of U.S. Provisional Application Ser. No. 60/510,351, filed on Oct. 10, 2003, and of U.S. Provisional Application Ser. No. 60/512,380, filed on Oct. 17, 2003. The entire contents of these provisional patent applications are hereby incorporated in their entirety by this reference.

### BACKGROUND OF THE INVENTION

Targets involved in the regulation of bone marrow development provide novel therapeutic approaches to the treatment of primary bone marrow failure and bone marrow dysfunction secondary to toxic insults, most notably chemotherapy-induced cytopenias. There is a severe unmet medical need in this arena as the few therapies currently available are recombinant proteins and all act at a relatively late stage of lineage differentiation.

Marrow populations of human and murine origin enriched for hematopoetic stem cells as well as bone marrow stromal cell populations provide useful sources of material for gene discovery and annotation of targets involved in proliferation and maturation of precursor populations. Hematopoietic cells cultured under various circumstances, isolated from humans in vivo, or from animal models in vivo provide a rich source of raw material for gene expression analysis leading to the identification of novel therapeutics useful for hematological disorders.

### DETAILED DESCRIPTION OF THE INVENTION

The present invention provides methods and compositions for the diagnosis and treatment of patients with hemtological disorders.

“Treatment”, as used herein, is defined as the application or administration of a therapeutic agent to a patient, or application or administration of a therapeutic agent to an isolated tissue or cell line from a patient, who has a disease or disorder, a symptom of disease or disorder or a predisposition toward a disease or disorder, with the purpose of curing, healing, alleviating, relieving, altering, remedying, ameliorating, improving or affecting the disease or disorder, at least one symptom of disease or disorder or the predisposition toward a disease or disorder. A therapeutic agent includes, but is not limited to, small molecules, peptides, antibodies, ribozymes and antisense oligonucleotides. Representative molecules are described herein.

A hematological disorder as used herein includes, but is not limited to erythroid-associated disorders. As used herein, the term “erythroid associated disorders” include disorders involving aberrant (increased or deficient) erythroblast proliferation, e.g., an erythroleukemia, and aberrant (increased or deficient) erythroblast differentiation, e.g., an anemia. Erythrocyte-associated disorders include anemias such as, for example, drug-(chemotherapy-) induced anemias, hemolytic anemias due to hereditary cell membrane abnormalities, such as hereditary spherocytosis, hereditary elliptocytosis, and hereditary pyropoikilocytosis; hemolytic anemias due to acquired cell membrane defects, such as paroxysmal nocturnal hemoglobinuria and spur cell anemia; hemolytic anemias caused by antibody reactions, for example to the RBC antigens, or antigens of the ABO system, Lewis system, Ii system, Rh system, Kidd system, Duffy system, and Kell system; methemoglobinemia; a failure of erythropoiesis, for example, as a result of aplastic anemia, pure red cell aplasia, myelodysplastic syndromes, sideroblastic anemias, and congenital dyserythropoietic anemia; secondary anemia in non-hematolic disorders, for example, as a result of chemotherapy, alcoholism, or liver disease; anemia of chronic disease, such as chronic renal failure; and endocrine deficiency diseases.

Agents that modulate the polypeptides of the present invention or nucleic acid activity or expression can be used to treat anemias, in particular, drug-induced anemias or anemias associated with cancer chemotherapy, chronic renal failure, malignancies, adult and juvenile rheumatoid arthritis, disorders of hemoglobin synthesis, prematurity, and zidovudine treatment of HIV infection. A subject receiving the treatment can be additionally treated with a second agent, e.g., erythropoietin, to further at least one symptom of the condition. The order of the treatments can be reversed. The two treatments can be administered simultaneously. The timing between treatments can be varied.

As used herein, the term “erythropoietin” or “EPO” refers to a glycoprotein produced in the kidney, which is the principal hormone responsible for stimulating red blood cell production (erythrogenesis). EPO stimulates the division and differentiation of committed erythroid progenitors in the bone marrow. Normal plasma erythropoietin levels range from 0.01 to 0.03 Units/mL, and can increase up to 100 to 1,000-fold during hypoxia or anemia. Graber and Krantz, Ann. Rev. Med. 29:51 (1978); Eschbach and Adamson, Kidney Intl. 28:1 (1985). Recombinant human erythropoietin (rHuEpo or epoietin alpha) is commercially available as EPOGEN.RTM. (epoietin alpha, recombinant human erythropoietin) (Amgen Inc., Thousand Oaks, Calif.) and as PROCRIT.RTM. (epoietin alpha, recombinant human erythropoietin) (Ortho Biotech Inc., Raritan, N.J.).

Another example of an erythroid-associated disorder is erythrocytosis. Erythrocytosis, a disorder of red blood cell overproduction caused by excessive and/or ectopic erythropoietin production, can be caused by cancers, e.g., a renal cell cancer, a hepatocarcinoma, and a central nervous system cancer. Diseases associated with erythrocytosis include polycythemias, e.g., polycythemia vera, secondary polycythemia, and relative polycythemia.

A hematological disorder as used herein includes disorders involving B-cells which include, but are not limited to precursor B-cell neoplasms, such as lymphoblastic leukemia/lymphoma. Peripheral B-cell neoplasms include, but are not limited to, chronic lymphocytic leukemia/small lymphocytic lymphoma, follicular lymphoma, diffuse large B-cell lymphoma, Burkitt lymphoma, plasma cell neoplasms, multiple myeloma, and related entities, lymphoplasmacytic lymphoma (Waldenstr\overline{o}m macroglobulinemia), mantle cell lymphoma, marginal zone lymphoma (MALToma), and hairy cell leukemia.

A hematological disorder as used herein includes disorders of the bone marrow which include but are not limited to: diseases involving hematopoeitic stem cells; committed lymphoid progenitor cells; lymphoid cells including B and T-cells; committed myeloid progenitors, including monocytes, granulocytes, and megakaryocytes; and committed erythroid progenitors. These include but are not limited to the leukemias, including B-lymphoid leukemias, T-lymphoid leukemias, undifferentiated leukemias; erythroleukemia, megakaryoblastic leukemia, monocytic; [leukemias are encompassed with and without differentiation; chronic and acute lymphoblastic leukemia, chronic and acute lymphocytic leukemia, chronic and acute myelogenous leukemia, lymphoma, myelo dysplastic syndrome, chronic and acute myeloid leukemia, myelomonocytic leukemia; chronic and acute myeloblastic leukemia, chronic and acute myelogenous leukemia, chronic and acute promyelocytic leukemia, chronic and acute myelocytic leukemia, hematologic malignancies of monocyte-macrophage lineage, such as juvenile chronic myelogenous leukemia; secondary AML, antecedent hematological disorder; refractory anemia; aplastic anemia; reactive cutaneous angioendotheliomatosis; fibrosing disorders involving altered expression in dendritic cells, disorders including systemic sclerosis, E-M syndrome, epidemic toxic oil syndrome, eosinophilic fasciitis localized forms of scleroderma, keloid, and fibrosing colonopathy; angiomatoid malignant fibrous histiocytoma; carcinoma, including primary head and neck squamous cell carcinoma; sarcoma, including kaposi's sarcoma; fibroadanoma and phyllodes tumors, including mammary fibroadenoma; stromal tumors; phyllodes tumors, including histiocytoma; erythroblastosis; neurofibromatosis; diseases of the vascular endothelium; demyelinating, particularly in old lesions; gliosis, vasogenic edema, vascular disease, Alzheimer's and Parkinson's disease; T-cell lymphomas; B-cell lymphomas.

A hematological disorder as used herein can include platelet disorders including but not limited to disorders related to reduced platelet number, thrombocytopenia, include idiopathic thrombocytopenic purpura, including acute idiopathic thrombocytopenic purpura, drug-induced thrombocytopenia, HIV-associated thrombocytopenia, and thrombotic microangiopathies: thrombotic thrombocytopenic purpura and hemolytic-uremic syndrome.

A hematological disorder can also include thrombosis. Thrombosis can result from platelet dysfunction, e.g. seen in myocardial infarction, angina, hypertension, lipid disorders, diabetes mellitus; myelodysplastic syndromes; myeloproliferative yndromes (including polycythemia vera and thombocythemia); thrombotic thrombocytopenic purpuras; HIV-induced platelet disorders (AIDS-Thrombocytopenia); heparin induced thrombocytopenia; mural cell alterations/interactions leading to platelet aggregation/degranulation, vascular endothelial cell activation/injury, monocyte/macrophage extravasation and smooth muscle cell proliferation; autoimmune disorders such as, but not limited to vasculitis, antiphospholipid syndromes, systemic lupus erythromatosis; inflammatory diseases, such as, but not limited to iImmune activation; graft Vs host disease; radiation induced hypercoagulation; clotting factor dysregulation either hereditary (autosomal dominant or recessive) such as, but not limited to clotting factor pathways including protein C/S, Anti-thrombin III deficiency, and the Factor V Leiden mutation or acquired such as but not limited to autoimmune, cancer-associated and drug-induced dysregulation of clotting factors.

A hematological disorder as used herein can include red cell disorders including but not limited to, anemias, such as hemolytic anemias, including hereditary spherocytosis, hemolytic disease due to erythrocyte enzyme defects: glucose-6-phosphate dehydrogenase deficiency, sickle cell disease, thalassemia syndromes, paroxysmal nocturnal hemoglobinuria, immunohemolytic anemia, and hemolytic anemia resulting from trauma to red cells; and anemias of diminished erythropoiesis, including megaloblastic anemias, such as anemias of vitamin B 12 deficiency: pernicious anemia, and anemia of folate deficiency, iron deficiency anemia, anemia of chronic disease, aplastic anemia, pure red cell aplasia, and other forms of marrow failure.

A hematological disorder as used herein can include disease of T cells including but not limited to, cell-mediated hypersensitivity, such as delayed type hypersensitivity and T-cell-mediated cytotoxicity, and transplant rejection; autoimmune diseases, such as systemic lupus erythematosus, Sjögren syndrome, systemic sclerosis, inflammatory myopathies, mixed connective tissue disease, and polyarteritis nodosa and other vasculitides; immunologic deficiency syndromes, including but not limited to, primary immunodeficiencies, such as thymic hypoplasia, severe combined immunodeficiency diseases, and AIDS; leukopenia; reactive (inflammatory) proliferations of white cells, including but not limited to, leukocytosis, acute nonspecific lymphadenitis, and chronic nonspecific lymphadenitis; neoplastic proliferations of white cells, including but not limited to lymphoid neoplasms, such as precursor T-cell neoplasms, such as acute lymphoblastic leukemia/lymphoma, peripheral T-cell and natural killer cell neoplasms that include peripheral T-cell lymphoma, unspecified, adult T-cell leukemia/lymphoma, mycosis fungoides and Sézary syndrome, and Hodgkin disease.

A hematological cell can include, but is not limited to a bone marrow cell, a hematopoeitic stem cell, an erythroid cell including a red blood cell, lymphoid cells including a B- and a T-cell, a myeloid (neutrophil) cell including a monocyte, a granulocyte, and a megakaryocyte, and a platelet.

One aspect of the invention features 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 polypeptides and biologically active or antigenic fragments thereof that are useful, e.g., as reagents or targets in assays applicable to treatment and diagnosis of 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 mediated or related disorders, e.g., hematopoietic disorders (e.g., erythroid associated disorders). In another embodiment, the invention provides 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 polypeptides having a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 activity.

In a related aspect, the invention provides 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 polypeptides or fragments operatively linked to non-9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 polypeptides to form fusion proteins.

In another aspect, the invention features antibodies and antigen-binding fragments thereof, that react with, or more preferably, specifically bind 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 polypeptides.

In another aspect, the invention provides methods of screening for compounds that modulate the expression or activity of the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 polypeptides or nucleic acids.

In still another aspect, the invention provides a process for modulating 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 polypeptide or nucleic acid expression or activity, e.g. using the screened compounds. In certain embodiments, the methods involve treatment of conditions related to decreased activity or expression of the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 polypeptides or nucleic acids, such as conditions involving aberrant cellular proliferation of a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014-expressing cell, e.g., a hematopoeitic cell (e.g., a myeloid (neutrophil) cell, a monocyte, an erythroid cell, a bone marrow cell, a CD34-expressing cell, a megakaryocyte). The condition may involve increased hematopoeitic cell activity or proliferation as in the case of leukemia, e.g., an erythroleukemia; or decreased hematopoietic cell differentiation as in the case of, e.g., an anemia.

In still another aspect, the invention features a method of modulating (e.g., enhancing or inhibiting) the proliferation, survival, and/or differentiation of a cell, e.g., a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014-expressing cell, e.g., a hematopoietic cell (e.g., a myeloid (neutrophil) cell, a monocyte, an erythroid cell, a bone marrow cell, a CD34-expressing cell, a megakaryocyte). The method includes contacting the cell with an agent that modulates the activity or expression of a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 polypeptide or nucleic acid, in an amount effective to modulate the proliferation and/or differentiation of the cell.

In a preferred embodiment, the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 polypeptide has an amino acid sequence identical to, or substantially identical to, SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78 or 80. In other embodiments, the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 polypeptide is a fragment of at least 15, 20, 50, 100, 150, 180, 200, or more contiguous amino acids of SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78 or 80.

In a preferred embodiment, the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 nucleic acid has a nucleotide sequence identical to, or substantially identical to, SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77 or 79. In other embodiments, the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 nucleic acid is a fragment of at least 50, 100, 150, 200, 250, 300, 350, 400, 450, 500, 550, 600, or more contiguous nucleotides of SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77 or 79.

In a preferred embodiment, an agent modulates (e.g., increases or decreases) expression of the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 nucleic acid by, e.g., modulating transcription, mRNA stability, etc.

In preferred embodiments, the agent is a peptide, a phosphopeptide, a small molecule, e.g., a member of a combinatorial library, or an antibody, or any combination thereof. The antibody can be conjugated to a therapeutic moiety selected from the group consisting of a cytotoxin, a cytotoxic agent and a radioactive metal ion.

In additional preferred embodiments, the agent is an antisense, a ribozyme, or a triple helix molecule, or a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 nucleic acid, or any combination thereof.

In a preferred embodiment, the agent is administered in combination with a cytotoxic agent.

In a preferred embodiment, the cell, e.g., the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014-expressing cell, is a hematopoietic cell, e.g., a myeloid, lymphoid or erythroid cell, or a precursor cell thereof. Examples of such cells include myelocytic cells (polymorphoneuclear cells), erythrocytic cells, lymphocytes, monocytes, reticular cells, plasma cells and megakaryocytes, as well as stem cells for the different lineages, and precursors for the committed progenitor cells, for example, precursors of red blood cells (erythroblasts), macrophages (monoblasts), platelets (megakaryocytes), polymorphoneuclear leucocytes (myeloblasts), and lymphocytes (lymphoblasts).

In a preferred embodiment, the cell, e.g., the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014-expressing cell, is a bone marrow cell, e.g., a bone marrow CD34-expressing cell. Examples of CD34-expressing cells include immature haematopoietic precursor cells, haematopoietic colony-forming cells in bone marrow, including unipotent (CFU-GM, BFU-E) and pluripotent progenitors (CFU-GEMM, CFU-Mix and CFU-blast); as well as stromal cell precursors, terminal deoxynucleotidyl transferase (TdT) expressing B- and T-lymphoid precursors, early myeloid cells and early erythroid cells.

In a preferred embodiment, the cell, e.g., the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014-expressing cell, is a bone marrow erythroid cell, e.g., an erythroid progenitor (e.g., a glycophorin A (GPA) (low)CD71+ cell) or a differentiated cell, e.g., an erythrocyte or a megakaryocyte.

In a preferred embodiment, the cell, e.g., the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014-expressing cell, is further contacted with a protein, e.g., a cytokine. Preferably, the protein is selected from the group consisting of G-CSF, GM-CSF, stem cell factor, and preferably erythropoietin. The protein contacting step can occur before, at the same time, or after the agent is contacted. The protein contacting step can be effected in vitro or ex vivo. For example, the cell, e.g., the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014-expressing cell is obtained from a subject, e.g., a patient, and contacted with the protein ex vivo. The treated cell can be re-introduced into the subject. Alternatively, the protein contacting step can occur in vivo.

In a preferred embodiment, the agent and the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014-polypeptide or nucleic acid are contacted in vitro or ex vivo.

In a preferred embodiment, the contacting step is effected in vivo in a subject, e.g., as part of a therapeutic or prophylactic protocol. Preferably, the subject is a human, e.g., a patient with a hematopoietic disorder, e.g., a leukemia or an erythroid-associated disorder. For example, the subject can be a patient with an anemia, e.g., hemolytic anemia, aberrant erythropoiesis, secondary anemia in non-hematolic disorders, anemia of chronic disease such as chronic renal failure; endocrine deficiency disease; and/or erythrocytosis (e.g., polycythemia). Alternatively, the subject can be a cancer patient, e.g., a patient with leukemic cancer, e.g., an erythroid leukemia, or a carcinoma, e.g., a renal carcinoma. In other embodiments, the subject is a non-human animal, e.g., an experimental animal.

The contacting step(s) can be repeated.

In a preferred embodiment, the agent decreases the proliferation and/or enhances the differentiation of the cell, e.g., the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014-expressing cell, e.g., the hematopoietic cell (e.g., the myeloid (neutrophil) cell, the monocyte, the erythroid cell, the bone marrow cell, the CD34-expressing cell, the megakaryocyte). Such agents can be used to treat or prevent cancers, e.g., leukemic cancers.

In a preferred embodiment, the agent increases the number of hematopoietic cells (e.g., myeloid (neutrophil) cells, monocytes, erythroid cells, bone marrow cells, CD34-expressing cells, megakaryocytes), by e.g., increasing the proliferation, survival, and/or stimulating the differentiation, of progenitor cells. Such agents can be used to treat or prevent hematopoietic disorders, e.g., anemias (e.g., hemolytic anemias, aberrant erythropoiesis, secondary anemias in nonhematolic disorders, anemias of chronic diseases such as chronic renal failure; endocrine deficiency diseases; and/or erythrocytosis, e.g., polycythemias).

In another aspect, the invention features a method of modulating hematopoiesis, e.g., erythropoiesis, comprising contacting a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014-expressing cell, e.g., a hematopoietic cell, (e.g., a myeloid (neutrophil) cell, a monocyte, an erythroid cell, a bone marrow cell, a CD34-expressing cell, a megakaryocyte), with a agent that increases or decreases the activity or expression of a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 polypeptide or nucleic acid, thereby modulating the differentiation of the hematopoietic cell.

In yet another aspect, the invention features a method of treating or preventing a hematopoietic disorder, e.g., an erythroid-associated disorder, in a subject. The method includes administering to the subject an effective amount of a agent that modulates the activity or expression of a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 polypeptide or nucleic acid such that the hematopoietic disorder is ameliorated or at least one symptom of the hematological disorder is decreased.

### MOLECULES OF THE PRESENT INVENTION

Gene ID 9118

The human 9118 sequence (SEQ ID NO:1), known also as chlordecone reductase homolog, is approximately 1175 nucleotides long including untranslated regions. The coding sequence, located at about nucleic acid 11 to 982 of SEQ ID NO:1, encodes a 323 amino acid protein (SEQ ID NO:2).

As assessed by TaqMan analysis, 9118 mRNA was expressed at high levels in CD34 progenitor cells, pooled erythroid cells and GPA high cells. Further TaqMan experiments indicated that 9118 mRNA was also expressed in liver, kidney, lung, spleen and fetal liver tissue samples. Within hematopoietic samples, 9118 mRNA expression was highest in erythroid cells followed by macrophages and progenitor cells.

9118 potentially plays a role in erythroid cell differentiation and proliferation. Therefore, inhibition of 9118 function would lead to increased proliferation with differentiation of erythroid cells from CFU-E to mature erythroid cells.

Due to the expression pattern of 9118 mRNA in CD34 progenitors, pooled erythroid cells and GPA high cells, along with its functional role, 9118 may play a role in regulating diseases associated with hematological disorders. Therefore, modulators of 9118 activity would be useful in treating hematological disorders. 9118 polypeptides of the current invention would be useful to screen for modulators of 9118 activity.

Gene ID 990

The human 990 sequence (SEQ ID NO:3), known also as tripeptidyl peptidase II, is approximately 4626 nucleotides long including untranslated regions. The coding sequence, located at about nucleic acid 24 to 3773 of SEQ ID NO:3, encodes a 1249 amino acid protein (SEQ ID NO:4).

As assessed by TaqMan analysis, 990 mRNA was expressed in fetal liver, Day 10 erythroid cultures and K562 erythroleukemia cell line. TaqMan analyses performed using erythroid panels demonstrated that 990 mRNA expression was high in GPA low cells and in Day 1-12 erythroid differentiation cultures. 990 mRNA expression was also elevated in BFU-E cultures, which are early erythroid progenitors. Further TaqMan analysis using an organ recital panel showed high expression of 990 mRNA in erythroid progenitors, maturing erythroid and megakaryocytic cells.

The mouse ortholog of 990 was shown to be expressed in lineage negative progenitor cells and in TER119+ erythroid cells.

990 mRNA is highly expressed in proliferating erythroid progenitors. 990 may function in growth factor or other protein degradation to control the proliferation rate of these cells. Inhibition of 990 in neurons leads to increased availability of CCK8 (Nature 1996; 380:403-409). 990 may also function to process proteins in the apoptotic pathway. Inhibition of 990 in infected macrophages leads to reduced apoptosis (Infect. Immun. 2000; 68:5502-5508). Therefore, inhibiting 990 may lead to expansion of erythroid cells.

Due to 990 mRNA expression in the fetal liver, Day 10 erythroid cultures and K562 erythroleukemia cell line, along with its functional role, modulators of 990 activity would be useful in treating hematological disorders. 990 polypeptides of the current invention would be useful to screen for modulators of 990 activity.

Gene ID 17662

The human 17662 sequence (SEQ ID NO:5), known also as vitellogenic carboxypeptidase like protein, is approximately 1638 nucleotides long including untranslated regions. The coding sequence, located at about nucleic acid 70 to 1500 of SEQ ID NO:5, encodes a 476 amino acid protein (SEQ ID NO:6).

As assessed by TaqMan analysis, 17662 mRNA was expressed in fetal liver, CD14−/CD19+ B-cells, CD34+ cells, GPA high cells, Day 10 erythroid cultures and stroma cells. Further TaqMan analyses performed using erythroid panels, demonstrated that 17662 mRNA expression was highest in GPA low cells and in Day 6 erythroid differentiation cultures. TaqMan analyses using mouse cell panels showed moderate expression of 17662 mRNA in all mouse cells tested, such as lineage negative mononuclear cells, TER119+ erythroid cells and GR-1+ myeloid cells. TaqMan analysis of an organ recital panel showed that 17662 mRNA expression was restricted to erythroid progenitors, maturing erythroid, neutrophilic cells, heart, kidney and bladder.

17662 is a carboxypeptidase and bears strong sequence similarity to serine carboxypeptidases. 17662 is highly expressed in proliferating erythroid progenitors. 17662 potentially plays a functional role in growth factors or other protein degradation pathways.

Due to 17662 mRNA expression in the fetal liver, CD14−/CD19+ B-cells, CD34+ cells, GPA high cells, Day 10 erythroid cultures and stroma cells, along with its functional role, 17662 may play a role in regulating diseases associated with hematological disorders. Therefore, modulators of 17662 activity would be useful in treating hematological disorders. 17662 polypeptides of the current invention would be useful to screen for modulators of 17662 activity.

Gene ID 81982

The human 81982 sequence (SEQ ID NO:7), known also as probable serine protease HTRA4 precursor, is approximately 1544 nucleotides long including untranslated regions. The coding sequence, located at about nucleic acid 87 to 1517 of SEQ ID NO:7, encodes a 476 amino acid protein (SEQ ID NO:8).

As assessed by TaqMan analysis, 81982 mRNA expression was restricted to the placenta, CD14−/CD11b−CD15+ neutrophil precursors, Day 10 neutrophil cultures, stroma cells and macrophages. Further TaqMan analysis indicated that 81982 mRNA was also expressed in early neutrophil precursors (CD14−,11B−,CD15+) and in Day 6 and 12 CD34 cells. TaqMan analysis using an organ recital panel showed high expresssion levels of 81982 mRNA in neutrophils with lesser expression in progenitor cells.

81982 mRNA is highly expressed in proliferating neutrophil cultures. 81982 may potentially play a functional role in growth factors or other protein degradation pathways. Inhibition of 81982 may lead to expansion of neutrophilic cells in vivo.

Due to 81982 mRNA expression in the placenta, CD14−/CD11b−CD15+ neutrophil precursors, Day 10 neutrophil cultures, stroma cells and macrophages, along with its functional role, 81982 may play a role in regulating diseases associated with hematological disorders. Therefore, modulators of 81982 activity would be useful in treating hematological disorders including but not limited to diseases characterized by neutropenia and/or an increase in the number of neutrophils. 81982 polypeptides of the current invention would be useful to screen for modulators of 81982 activity.

Gene ID 630

The human 630 sequence (SEQ ID NO:9), known also as voltage-gated potassium channel KCNA3, is approximately 3303 nucleotides long including untranslated regions. The coding sequence, located at about nucleic acid 381 to 1952 of SEQ ID NO:9, encodes a 523 amino acid protein (SEQ ID NO:10).

As assessed by TaqMan analysis, 630 mRNA was highly expressed in megakaryocytes generated in vitro. Further TaqMan analyses indicated that 630 mRNA was expressed at high levels in CD3 and CD4 T-cells and in brain. 630 mRNA was expressed at high levels in the platelets of patients with coronary artery disease and in platelets from normal volunteers.

The voltage-gated potassium channel, KCNA3 or 630, is known to be expressed in lymphocytes (PNAS 1990; 87:9411-9415). Expression data reveals high levels of 630 mRNA in platelets and megakaryocytes. The role of 630 in platelet aggregation was tested by using a known channel blocker charybdotoxin, which inhibits KCNA3. Experiments revealed that charybdodoxin inhibits both thrombin-dependent calcium flux and aggregation in human platelets. Based on in vitro studies and the expression profile, inhibition of 630 would inhibit platelet aggregation and thrombus formation in vivo.

Due to the expression pattern of 630 in megakaryocytes generated in vitro, along with its functional role, 630 may play a role in regulating diseases associated with hematological disorders. Therefore, modulators of 630 activity would be useful in treating hematological disorders. 630 polypeptides of the current invention would be useful to screen for modulators of 630 activity.

Gene ID 21472

The human 21472 sequence (SEQ ID NO:11), known also as NADP-dependent retinol dehydrogenase/reductase, is approximately 960 nucleotides long. The coding sequence, located at about nucleic acid 1 to 960 of SEQ ID NO:11, encodes a 319 amino acid protein (SEQ ID NO:12).

As assessed by TaqMan analysis, 21472 mRNA expression was very restricted, with expression levels higher in mature neutrophils (CD14+CD15+CD11b+) when compared to immature neutrophils (CD14+CD15+CD11B−). 21472 mRNA was also expressed in differentiating neutrophils and resting macrophages. TaqMan analysis using an organ recital panel indicated that 21472 mRNA was highly expressed in tissues with significant neutrophil composition, such as colon tissues with tumor or inflammatory bowel disease and tonsil tissues.

21472 is involved in the first step of retinol biosynthesis. Retinoic acid is known to induce differentiation in myeloid progenitors and end the proliferative cycle. Therefore, inhibiting retinoic acid production would lead to increased numbers of myeloid progenitors.

Due to its functional role and expression pattern in the mature neutrophil, 21472 plays a role in regulating diseases associated with hematological disorders. Therefore, modulators of 21472 activity would be useful in treating hematological disorders including but not limited to diseases characterized by neutropenia and/or an increase in number of neutrophils. 21472 polypeptides of the current invention would be useful to screen for modulators of 21472.

Gene ID 17692

The human 17692 sequence (SEQ ID NO:13), known also as Carboxypeptidase CPX-1, is approximately 2205 nucleotides long. The coding sequence, located at about nucleic acid 1 to 2205 of SEQ ID NO:13, encodes a 734 amino acid protein (SEQ ID NO:14).

As assessed by TaqMan analysis, 17692 mRNA expression was restricted to CD34+ progenitor cells. Further TaqMan analyseis indicated that 17692 mRNA was expressed in early erythroid, megakaryocyte and neutrophil cultures, but was down regulated with differentiation in all lineages.

Carboxypeptidases are known to degrade other peptides. Scientific literature indicates that carboxypeptidases modulate growth by degrading growth factors and cytokines. The highly restricted expression of 17692 in CD34+ progenitor cells in vivo indicates that 17692 helps control cellular growth and differentiation by degrading stimulatory growth factors. Therefore, inhibiting 17692 will lead to the stimulation of hematopoiesis.

Due to 17692 mRNA expression in CD34+ progenitor cells, along with its functional role, modulators of 17692 would be useful in the treatment of hematological disorders. 17692 polypeptides of the present invention would be useful in screening for modulators of 17692 activity.

Gene ID 19290

The human 19290 sequence (SEQ ID NO:15), known also as multiple inositol polyphosphate phosphatase, is approximately 2416 nucleotides long including untranslated regions. The coding sequence, located at about nucleic acid 23 to 1486 of SEQ ID NO:15, encodes a 487 amino acid protein (SEQ ID NO:16).

As assessed by TaqMan analysis, 19290 mRNA was expressed in erythroid lineages, GPA high cells, GPA low cells and fetal liver.

19290 is an inositol polyphosphate phosphatase. 19290 degrades insitol 6P (Ins6P) and Ins5P. Ins6P and Ins5P are known to play important roles in signal transduction resulting in the proliferation of a number of cell types. Therefore, inhibiting 19290 will stimulate proliferation of erythroid cells by reducing the degradation of the inositol phosphates. This will result in a larger number of erythroid cells, thereby being an effective treatment for anemia.

Due to 19290 mRNA expression in erythroid lineages, along with its functional role, modulators of 19290 would be useful in the treatment of hematological disorders. 19290 polypeptides of the present invention would be useful in screening for modulators of 19290 activity.

Gene ID 21620

The human 21620 sequence (SEQ ID NO:17), known also as a short-chain dehydrogenase/reductase (SDR; alcohol dehydrogenase), is approximately 1909 nucleotides long including untranslated regions. The coding sequence, located at about nucleic acid 421 to 1203 of SEQ ID NO:17, encodes a 260 amino acid protein (SEQ ID NO:18).

As assessed by TaqMan analysis, 21620 mRNA was expressed at high levels in cells of erythroid lineage, GPA low cells and in cultured erythroid cells. 21620 mRNA expression was also observed in fetal liver and GPA hi cells.

Alcohol dehydrogenases are responsible for metabolism of retinoids and steroid hormones, which are known to have adverse effects on hematopoiesis. Therefore, inhibiting 21620 will reduce the amount of retinoids and steroid hormones in the bone marrow, thereby stimulating hematopoiesis.

Due to 21620 mRNA expression in erythroid cells, along with its functional role, modulators of 21620 would be useful in the treatment of hematological disorders. 21620 polypeptides of the present invention would be useful in screening for modulators of 21620 activity.

Gene ID 21689

The human 21689 sequence (SEQ ID NO:19), known also as TWIK-related acid-sensitive K+ channel 2 (TASK2), is approximately 3514 nucleotides long including untranslated regions. The coding sequence, located at about nucleic acid 340 to 1839 of SEQ ID NO:19, encodes a 499 amino acid protein (SEQ ID NO:20).

As assessed by TaqMan analysis, 21689 mRNA was expressed at high levels in cells of erythroid and myeloid lineages. 21689 mRNA expression increased significantly during erythroid differentiation and was moderately expressed (and maintained) during myeloid differentiation. 21689 mRNA was also expressed in hematopoietic progenitor cells and in GPA low cells.

21689 is also known as TASK2, an outwardly rectifying potassium channel. These channels are known to be important in maintaining cell membrane currents and function in a variety of cellular processes. By agonizing 21689, potassium will flow out of the cells resulting in a change in membrane potential on differentiating erythroid cells. The change in membrane potential will result in a stimulation of proliferation, which leads to increased numbers of erythrocytes in the periphery and therefore would be an effective treatment for anemia.

Due to 21689 mRNA expression in erythroid cells, along with its functional role, modulators of 21689 would be useful in the treatment of hematological disorders. 21689 polypeptides of the present invention would be useful in screening for modulators of 21689 activity.

Gene ID 28899

The human 28899 sequence (SEQ ID NO:21), known also as lysophosphatidic acid acyltransferase-gamma (LPAAT-gamma), is approximately 1832 nucleotides long including untranslated regions. The coding sequence, located at about nucleic acid 192 to 1322 of SEQ ID NO:21, encodes a 376 amino acid protein (SEQ ID NO:22)

As assessed by TaqMan analysis, 28899 mRNA was expressed during erythroid differentiation. Specifically, expression of 28899 mRNA was high in erythroid cells as they differentiate in vitro, increasing at days 6 and 12 in culture. Expression of 28899 mRNA was highest in GPA low and GPA high cells in vivo. Expression of 28899 mRNA was also high in fetal liver, an erythropoietic organ.

1-acyl-sn-glycerol-3-phosphate acyltransferases (AGPATs) convert lysophosphatidic acid (LPA) to phosphatidic acid (PA) in a variety of cell types. LPA is a regulator of cellular proliferation. Therefore, 28899 is potentially involved in regulating neutrophil proliferation/differentiation decisions. Antagonizing 28899 allows for the expansion of a pool of erythroid cells which can replenish erythroid cells depleted in anemia.

Due to 28899 mRNA expression in erythroid cells, along with its functional role, modulators of 28899 would be useful in the treatment of hematological disorders. 28899 polypeptides of the present invention would be useful in screening for modulators of 28899 activity.

Gene ID 53659

The human 53659 sequence (SEQ ID NO:23), known also as OATP-H or OATP-M1, is approximately 2634 nucleotides long including untranslated regions. The coding sequence, located at about nucleic acid 104 to 2278 of SEQ ID NO:23, encodes a 724 amino acid protein (SEQ ID NO:24).

As assessed by TaqMan analysis, 53659 mRNA was expressed in cells of myeloid lineage, CD11b-CD15+ cells, CD11b+CD15+ cells, kidney and liver. 53659 mRNA was also expressed in BFU erythroid cells and cultured neutrophils.

53659 is a prostaglandin transporter. Prostaglandins are known to have both positive and negatives effects on hematopoiesis. By agonizing 53659, the inhibitory effects of prostaglandins on cells of myeloid lineage would decrease. This would result in larger numbers of differentiating myeloid cells, and therefore, be an effective treatment for chemotherapy-induced neutropenia.

Due to 53659 mRNA expression in myeloid lineage cells, CD11b-CD15+ cells, CD11b+CD15+ cells, kidney and liver, along with its functional role, modulators of 53659 would be useful in the treatment of hematological disorders. 53659 polypeptides of the present invention would be useful in screening for modulators of 53659 activity.

Gene ID 64549

The human 64549 sequence (SEQ ID NO:25), known also as Narc16b, is approximately 2019 nucleotides long. The coding sequence, located at about nucleic acid 1 to 2019 of SEQ ID NO:25, encodes a 672 amino acid protein (SEQ ID NO:26).

As assessed by TaqMan analysis, 64549 mRNA was expressed at very high levels in cells of erythroid lineage. In cultured human cells, 64549 mRNA expression increased with differentiation, with the highest expression seen in GPA high and GPA low cells. Expression of 64549 mRNA was also seen in fetal liver.

The Narc family of genes are glycerophosphodiesterases that are known to be involved in signaling, potentially through glyceraldehyde-3-phosphate dehydrogenase (GAPDH). Their over expression in cultured neuronal cells and primary neuronal cells is known to cause apoptosis. Inhibiting Narc expression decreases induced apoptosis. By inhibiting 64549 in erythroid cells, apoptosis of differentiating erythroid cells would decrease. This would result in larger numbers of erythroid cells.

Due to 64549 mRNA expression in erythroid cells, along with its functional role, modulators of 64549 would be useful in the treatment of hematological disorders. 64549 polypeptides of the present invention would be useful in screening for modulators of 64549 activity.

Gene ID 9465

The human 9465 sequence (SEQ ID NO:27), known also as ATP-sensitive inward rectifier potassium channel 12, is approximately 2213 nucleotides long including untranslated regions. The coding sequence, located at about nucleic acid 451 to 1752 of SEQ ID NO:27, encodes a 433 amino acid protein (SEQ ID NO:28).

As assessed by TaqMan analysis, 9465 mRNA expression was restricted to erythroid lineage cells.

The inward rectifier potassium channels are characterized by their tendency to allow potassium to flow into a cell. Inhibition of potassium channels has been shown to result in decreased cellular proliferation. Opening 9465 would increase intracellular potassium levels leading to increased proliferation of erythroid lineage cells.

Due to 9465 mRNA expression in erythroid cells, along with its functional role, modulators of 9465 would be useful in the treatment of hematological disorders. 9465 polypeptides of the present invention would be useful in screening for modulators of 9465 activity.

Gene ID 23544

The human 23544 sequence (SEQ ID NO:29), known also as cell cycle protein p38-2G4 homolog (hG4-1), is approximately 1697 nucleotides long including untranslated regions. The coding sequence, located at about nucleic acid 98 to 1282 of SEQ ID NO:29, encodes a 394 amino acid protein (SEQ ID NO:30).

As assessed by TaqMan analysis, 23544 mRNA was expressed in erythroid lineage cells and fetal liver. Expression of 23544 mRNA was higher in earlier progenitors than in later cells.

Proteases have been shown to degrade growth factors and other factors essential for hematopoiesis. Inhibition of 23544 potentially blocks growth factor degradation and enhances hematopoiesis, particularly in erythroid lineage cells.

Due to 23544 mRNA expression in erythroid cells, along with its functional role, modulators of 23544 would be useful in the treatment of hematological disorders. 23544 polypeptides of the present invention would be useful in screening for modulators of 23544 activity.

Gene ID 7366

The human 7366 sequence (SEQ ID NO:31), known also as chloride channel protein 4 (ClC4), is approximately 4454 nucleotides long including untranslated regions. The coding sequence, located at about nucleic acid 276 to 2558 of SEQ ID NO:31, encodes a 760 amino acid protein (SEQ ID NO:32).

As assessed by TaqMan analysis, 7366 mRNA expression was high in cells of erythroid and megakaryocyte lineages. 7366 mRNA expression was low in organs, except for brain and fetal liver tissue samples. Expression of 7366 mRNA increased in erythroid cells in vivo and was highest in GPA low cells. Expression of 7366 mRNA also increased with erythroid and megakaryocyte differentiation in vitro.

Chloride channels are known to be involved in a wide variety of cellular processes and can regulate such basic functions as cell volume and intracellular pH. Chloride channels have also been shown to play an essential role in proliferation. Inhibition of chloride channels can significantly decrease their proliferation. Agonizing 7366 would increase cellular proliferation. Agonizing 7366 in early erythroid cells would lead to an increased number of red blood cells, and therefore be an effective treatment for anemia.

Due to 7366 mRNA expression in erythroid and megakaryocyte lineages, along with its functional role, modulators of 7366 would be useful in the treatment of hematological disorders. 7366 polypeptides of the present invention would be useful in screening for modulators of 7366 activity.

Gene ID 27417

The human 27417 sequence (SEQ ID NO:33), known also as lysophosphatidic acid acyltransferase-epsilon (LPAAT-epsilon), is approximately 3725 nucleotides long including untranslated regions. The coding sequence, located at about nucleic acid 306 to 1400 of SEQ ID NO:33, encodes a 364 amino acid protein (SEQ ID NO:34).

As assessed by TaqMan analysis, 27417 mRNA was expressed at high levels in fetal liver and GPA low cells. 27417 mRNA was expressed at moderate levels in CD34+ cells, neutrophils, K562 and HL60 cell lines. Further TaqMan analysis indicated that 27417 mRNA expression increased with erythroid differentiation in vitro, with the highest expression levels at day 12.

27417 is also known as LPAAT-epsilon. These enzymes are known to catalyze the conversion of LPA to PA. LPA is known to stimulate proliferation in a variety of cell types. By inhibiting expression of 27417, higher levels of LPA are obtainable, leading to increased proliferation rates. Inhibition of 27417 in cells of erythroid lineage would increase the numbers of red blood cells, and therefore be an effective treatment for anemia.

Due to 27417 mRNA expression CD34+ cells, neutrophils, K562, and HL60 cell lines, along with its functional role, modulators of 27417 would be useful in the treatment of hematological disorders. 27417 polypeptides of the present invention would be useful in screening for modulators of 27417 activity.

Gene ID 57259

The human 57259 sequence (SEQ ID NO:35), known as an organic ion (sugar) transporter, is approximately 1855 nucleotides long including untranslated regions. The coding sequence, located at about nucleic acid 377 to 1594 of SEQ ID NO:35, encodes a 405 amino acid protein (SEQ ID NO:36).

As assessed by TaqMan analysis, 57259 mRNA was highly expressed in vivo in cells and tissues of erythroid lineage, including fetal liver. Moderate expression of 57259 mRNA was also seen in brain and kidney. Expression of 57259 mRNA was equally high in GPA low and GPA high erythroid cells in vitro.

57259 is an organic ion transporter. These proteins are known to transport a variety of organic molecules, including sugars and prostaglandins. Such molecules play crucial regulatory roles in the growth and proliferation of cells of erythroid lineage. Therefore, perturbing the activity of 57259 will result in greater proliferation of cells of erythroid lineage. This would result in greater numbers of red blood cells, and therefore would be an effective treatment for anemia.

Due to 57259 mRNA expression in erythroid cells, along with its functional role, modulators of 57259 would be useful in the treatment of hematological disorders. 57259 polypeptides of the present invention would be useful in screening for modulators of 57259 activity.

Gene ID 21844

The human 21844 sequence (SEQ ID NO:37), known also as 2-amino-3-ketobutyrate coenzyme A ligase, is approximately 1453 nucleotides long including untranslated regions. The coding sequence, located at about nucleic acid 4 to 1263 of SEQ ID NO:37, encodes a 419 amino acid protein (SEQ ID NO:38).

As assessed by TaqMan analysis, 21844 mRNA was highly expressed in the brain cortex, heart, human umbilical vein endothelial cells (HUVEC), pancreas and erythroid cells. Further TaqMan analysis indicated that in vitro, 21844 mRNA was expressed at the highest levels in erythroid cells. 21844 was also highly expressed by proliferating megakaryocytes, however expression in these cells decreased upon differentiation to mature cells. 21844 mRNA expression was also seen in fetal liver, which has a large proportion of erythroid cells. 21844 mRNA was expressed at much lower levels in T-cells, myeloid cells and megakaryocytes.

21844 is also known as 2-amino-3-ketobutyrate coenzyme A ligase. 21844 is involved in conversion of L-threonine to glycine. An antisense strategy showed that amino-ketobutyrate ligase is involved in the proliferation arrest of NSCLC-N6 cells in G1-phase after VT1 treatment (Anticancer Res 2002 Jul.-Aug.; 22(4):2229-35). Therefore inhibiting 21844 function will lead to increased cellular proliferation.

Due to 21844 mRNA expression in erythroid cells, along with its functional role, modulators of 21844 would be useful in the treatment of hematological disorders. 21844 polypeptides of the present invention would be useful in screening for modulators of 21844 activity.

Gene ID 943

The human 943 sequence (SEQ ID NO:39), known also as protein tyrosine phosphatase MEG1 (PTPMEG1), is approximately 3643 nucleotides long including untranslated regions. The coding sequence, located at about nucleic acid 772 to 3552 of SEQ ID NO:39, encodes a 926 amino acid protein (SEQ ID NO:40).

As assessed by TaqMan analysis, 943 mRNA was expressed at high levels in erythroid cells, with the highest expression observed in GPA low cells, GPA high cells and Day 10 in vitro cultured erythroid cells. 943 mRNA expression was also observed in fetal liver, brain, skeletal muscle, kidney and T-cells. 943 mRNA was up regulated with differentiation of erythroid cells cultured in vitro. Up regulation of 943 mRNA was also evident in in vitro BFU-E cultures in response to EPO stimulation. 943 mRNA expression observed in GPA low cells was maintained in GPA high cells, which is consistent with the fact that levels of the EPO receptor are also not regulated in these particular samples. Finally, 943 mRNA expression was observed in lineage negative and TER119+ cells obtained from mice.

943, also known as PTPMEG1 was cloned from megakaryocytes and has been described to interact with the glutamate receptor delta 2. Over expression of 943 was shown to slow the growth of COS cells (PNAS U.S.A. 1996; 93(23):12980-5). Based in part on the high expression of 943 in erythroid cells and its differential expression during the differentiation of erythroid cells, inhibition of 943 would result in increased proliferation of erythroid cells and their progenitors. Therefore 943 can be used to treat hematological disorders.

Due to 943 mRNA expression, along with its functional role, modulators of 943 would be useful in the treatment of hematological disorders. 943 polypeptides of the present invention would be useful in screening for modulators of 943 activity.

Gene ID 2061

The human 2061 sequence (SEQ ID NO:41), known also as serine/threonine protein kinase 25 (Ste20, yeast homolog) or SOK1, is approximately 1975 nucleotides long including untranslated regions. The coding sequence, located at about nucleic acid 127 to 1407 of SEQ ID NO:41, encodes a 426 amino acid protein (SEQ ID NO:42).

As assessed by TaqMan analysis, 2061 mRNA was most highly expressed in erythroid cells, skeletal muscle, HUVECs and brain cortex. TaqMan analysis of an hematopoietic cell panel showed 2061 mRNA was also highly expressed in GPA low cells, pooled Day 10 differentiating erythroid cells, K562 erythroleukemia cells and HUVECs. 2061 mRNA was also expressed in fetal liver. 2061 mRNA expression was low in T-cells, myeloid cells and progenitors.

In vitro, the levels of 2061 mRNA expression increased as erythroid cells differentiated, with the highest expression levels occurring at day 6, which corresponds to the peak of CFU-E activity. Expression levels decrease again by day 12 cultures. 2061 mRNA expression levels also increased in BFU-E cultures in the presence of EPO. 2061 mRNA expression was detected in GPA low and GPA high samples.
mRNA expression of the mouse 2061 ortholog was detected in TER119+ positive erythroid cells and in lineage negative fractions of bone marrow corresponding to the progenitor cell population. It was also detectable in bone marrow white blood cells, which contain lineage negative cells, and in Gr-1+ neutrophils.

2061 is a human homolog of yeast STE20 from the GC kinase group. It has been identified as a stress response kinase. SOK-1 is activated 3- to 7-fold by reactive oxygen intermediates (EMBO J. 1996 Sep. 2; 15(17):4537-46). The activation of SOK-1 may be one of the cells earliest responses to inducers of necrotic cell death (J. Biol. Chem. 1997 Nov. 14; 272(46):29372-9). Inhibition of SOK-1 function would lead to protection from cell death and apoptosis at the CFU-E stage and lead to increased numbers of erythroid cells.

Due to 2061 mRNA expression, along with its functional role, modulators of 2061 would be useful in the treatment of hematological disorders. 2061 polypeptides of the present invention would be useful in screening for modulators of 2061 activity.

Gene ID 5891

The human 5891 sequence (SEQ ID NO:43), known also as prolyl endopeptidase (PEP), is approximately 2562 nucleotides long including untranslated regions. The coding sequence, located at about nucleic acid 1 to 2133 of SEQ ID NO:43, encodes a 710 amino acid protein (SEQ ID NO:44).

As assessed by TaqMan analysis, 5891 mRNA was highly expressed in erythroid cells, HUVECs, brain cortex, and skeletal muscle. Further TaqMan analysis showed that the highest levels of 5891 mRNA expression were observed in pooled erythroid Day 10 cultures, K562 erythroleukemia cells, and in HUVECs. There was detectable, but low, expression in CD34 progenitor cells and T-cells. During differentiation of erythroid cells in vitro, expression of 5891 mRNA increased gradually from 24 hours onwards and peaked in Day 6 cultures (corresponding to CFU-Es). Consistent with the high expression levels seen in erythroid progenitors, 5891 mRNA expression in the proliferation competent GPA low cells was increased when compared to the more differentiated GPA high cells.

Expression of the mouse ortholog of 5891 was detected in mouse bone marrow cell fractions including lineage negative progenitor cells and erythroid TER119+ cells.

Cloned from T-cells, prolyl endopeptidase is known to cleave peptide bonds on the C-terminal side of prolyl residues. It is thought to play an important role in the degradation of peptide hormones and neuropeptides. (Gene 1994; 149(2), 363-366; J. Biochem 1994; 115(4), 724-729). It has been suggested that prolyl endopeptidase is involved in induction of apoptotic cell death in T-cell progenitors. Inhibitors of this enzyme protect the cells from apoptosis (FEBS Lett. 2002 Feb. 13; 512(1-3):163-7). Inhibition of this enzyme would lead to increased survival of erythroid progenitors, leading to increased numbers of erythroid cells.

Due to 5891 mRNA expression, along with its functional role, modulators of 5891 would be useful in the treatment of hematological disorders. 5891 polypeptides of the present invention would be useful in screening for modulators of 5891 activity.

Gene ID 9137

The human 9137 sequence (SEQ ID NO:45), known also as aldehyde dehydrogenase 7 (ALDH7), is approximately 2790 nucleotides long including untranslated regions. The coding sequence, located at about nucleic acid 48 to 1454 of SEQ ID NO:45, encodes a 468 amino acid protein (SEQ ID NO:46).

As assessed by TaqMan analysis, 9137 mRNA was expressed in many tissues and organs, but was most highly expressed in erythroid cells, neutrophils, lung, brain and kidney. 9137 mRNA expression in hematopoietic cells was restricted to CD11b−, CD15+ neutrophils and in vitro differentiating Day 10 cultures, CD14+ monocytes, GPA low erythroid cells and pooled Day 10 erythroid cultures. 9137 mRNA expression in erythroid cells increased during in vitro differentiation, especially at day 6 and day 12. Similarly, 9137 mRNA expression increased in BFU-E cultures with EPO. 9137 mRNA expression levels were higher in immature GPA low fractions when compared to the more mature GPA high fractions. In myeloid cells, 9137 mRNA expression also increased with myeloid differentiation in vitro but the levels were lower than in erythroid cells. 9137 mRNA expression was higher in the more immature CD11b− neutrophils than in the CD11b+ neutrophils (all CD14−, CD15+).

Expression of the mouse ortholog of 9137 was detectable in hematopoietic cells, with the highest levels of expression observed in TER119+ erythroid cells and GR-1+ neutrophils.

Twelve aldehyde dehydrogenase (ALDH) genes have been identified in humans. These genes, located on different chromosomes, encode a group of enzymes that oxidizes varieties of aliphatic and aromatic aldehydes. Comparison of the human ALDHs indicates a wide range of divergency (from <15% to >80% identity at the protein sequence level) among them (Gene. 1994 Dec. 30; 151(1-2):285-9).

The products of ALDH7 lead to the induction of differentiation of erythroid and myeloid progenitors. Inhibition of the function of this enzyme will lead to decreased differentiation and increased proliferation of myeloid and erythroid progenitors.

Due to 9137 mRNA expression, along with its functional role, modulators of 9137 would be useful in the treatment of hematological disorders. 9137 polypeptides of the present invention would be useful in screening for modulators of 9137 activity.

Gene ID 13908

The human 13908 sequence (SEQ ID NO:47), known also as serine protease HTRA1, is approximately 2036 nucleotides long including untranslated regions. The coding sequence, located at about nucleic acid 49 to 1491 of SEQ ID NO:47, encodes a 480 amino acid protein (SEQ ID NO:48).

As assessed by TaqMan analysis, 13908 mRNA was most highly expressed in synovium, which is consistent with the presence of stromal cells. 13908 mRNA was also expressed in HUVECs, ovary, breast, vein and lung. There was no 13908 mRNA expression in hematopoietic progenitor or mature cells. 13908 mRNA expression was restricted to primary bone marrow stromal cell samples, MF11 and MF12, cultured in vitro.

The mouse ortholog of 13908 was not expressed in hematopoietic cells but expression was detected in total bone marrow samples that contain stromal cells.

The bone marrow stroma produces growth factors to support the survival and proliferation of progenitor cells. Regulation of this system may involve the stroma producing proteases to degrade excess growth factors to protect the stem cell pool from excessive proliferation (Blood. 2001 Nov. 1; 98(9):2697-706.) HTRA1 has been reported to be a secreted protease, originally described as interacting with insulin growth factor (IGF) binding proteins, however the exact function of this protease is unknown (FEBS Lett. 1996 Dec. 2; 398(2-3):187-92.) Over expression has been demonstrated to inhibit proliferation of melanoma cells and its down regulation is associated with melanoma progression (Oncogene. 2002 Sep. 26; 21(43):6684-8.) HTRA2, a family member, has been reported to degrade inhibitors of apoptosis proteins (IAPs). HTRA1 is highly expressed in stromal cells. Therefore, 13908 may function in growth factor processing or other protein degradation to control the proliferation rate of the surrounding bone marrow progenitor cells. Inhibiting its function would lead to expansion of megakaryocytic, neutrophil and erythroid cells in vivo.

Due to 13908 mRNA expression in erythroid cells, along with its functional role, modulators of 13908 would be useful in the treatment of hematological disorders. 13908 polypeptides of the present invention would be useful in screening for modulators of 13908 activity.

Gene ID 14310

The human 14310 sequence (SEQ ID NO:49), known also as acetyl-coenzyme A acetyltransferase 2 (ACAT2), is approximately 1490 nucleotides long including untranslated regions. The coding sequence, located at about nucleic acid 38 to 1231 of SEQ ID NO:49, encodes a 397 amino acid protein (SEQ ID NO:50).

As assessed by TaqMan analysis, 14310 mRNA showed restricted expression. 14310 mRNA was highly expressed in erythroid cells, progenitor cells and brain. 14310 mRNA was expressed at much lower levels in other tissues and organs. 14310 mRNA was expressed in a restricted manner in hematopoietic cells, with the highest expression in pooled Day 10 erythroid cultures, megakaryocytic cultures and K562 erythroleukemia cells. Lower expression levels of 14310 mRNA were observed in CD34+ progenitors (which also contain erythroid progenitors). Upon differentiation of erythroid cells in vitro, the expression of 14310 mRNA increased, peaking at day 6, which corresponds to the highest erythroid specific proliferation potential of the cells. 14310 mRNA was also expressed at higher levels in GPA low cells (immature cells) compared to the more mature GPA high cells. These data suggest that this enzyme has a role in erythroid progenitors.

Mouse hematopoietic cells express the ortholog of 14310. The highest mRNA expression levels were observed in TER119+ erythroid cells. Lineage negative cells, which contain the erythroid progenitors, also expressed 14310.

ACAT2 is an enzyme involved in lipid metabolism. The function of ACAT2 has been studied in relation to atherosclerosis and a decreased level of cholesterol ester was noted (Proc Natl Acad Sci USA. 2003 Feb. 4; 100(3): 1262-7. Epub 2003 Jan. 21). It has not been examined in hematopoietic cells. It is notable that null mice are viable and healthy. ACAT2 is involved in the production of lipids that limit erythroid proliferation and promote differentiation. Inhibition of this enzyme would lead to more cells proliferating and ultimately lead to increased erythroid cell numbers.

Due to 14310 mRNA expression, along with its functional role, modulators of 14310 would be useful in the treatment of hematological disorders. 14310 polypeptides of the present invention would be useful in screening for modulators of 14310 activity.

Gene ID 17600

The human 17600 sequence (SEQ ID NO:51), known also as histone acetyltransferase type B catalytic subunit, is approximately 1568 nucleotides long including untranslated regions. The coding sequence, located at about nucleic acid 37 to 1296 of SEQ ID NO:51, encodes a 419 amino acid protein (SEQ ID NO:52).

As assessed by TaqMan analysis, 17600 mRNA was expressed almost exclusively in hematopoietic cells, with the highest expression levels in vitro in differentiated Day 10 erythroid cultures. 17600 mRNA was also significantly expressed in K562 cells and GPA low erythroid cells. 17600 mRNA expression increased with the differentiation of erythroid cells in vitro. Elevated expression levels of 17600 mRNA were observed in GPA low erythroid cells when compared to the more mature GPA high cells. 17600 expression was observed in lineage negative and TER 119+ cells, which correspond to erythroid progenitors and mature cells. In the mouse EPO model, expression of 17600 mRNA increased in samples treated with EPO when compared to controls.

It is the present invention that inhibitors of 17600 can be used to treat hematopoietic disorders. This is based, in part, on the studies first described herein and previous work on this molecule. 17600 is a histone acetyltransferase and histone acetylation has been linked to gene activation. (Cell. 1996; 84(6):843-851). Histone deacetylase inhibitors are currently being developed as anti-proliferative anti-cancer agents. Since histone acetylases work in the opposite direction from histone deacetylase, they could potentially inhibit the proliferative responses. The studies described herein show differential regulation in erythroid cells undergoing differentiation. It is therefore believed that an inhibitor of 17600 will lead to increased proliferation of erythroid progenitor cells by inhibiting the activation of genes that are involved in preventing the proliferation of erythroid progenitors resulting in more erythroid cells. Thus, the present invention could be used to ameliorate hematological disorders.

Due to 17600 mRNA expression, along with its functional role, modulators of 17600 would be useful in the treatment of hematological disorders. 17600 polypeptides of the present invention would be useful in screening for modulators of 17600 activity.

Gene ID 25584

The human 25584 sequence (SEQ ID NO:53), known also as aminoacylase 1, is approximately 1415 nucleotides long including untranslated regions. The coding sequence, located at about nucleic acid 62 to 1288 of SEQ ID NO:53, encodes a 408 amino acid protein (SEQ ID NO:54).

As assessed by TaqMan analysis, 25584 mRNA was most highly expressed in erythroid cells and kidney. There was detectable but lower expression in mouse organs and tissues. 25584 mRNA expression in hematopoietic cells was restricted to pooled Day 10 erythroid cultures and K562 cells with lower expression in CD34 cells. Expression of 25584 mRNA in erythroid cells was highest in GPA low cells and at day 6 of erythroid cells differentiating in vitro. These represent the proliferating erythroid cells that express high levels of the EPO receptor. Similarly, 25584 mRNA expression levels decreased as the cells mature to GPA high cells or Day 12 cultures in vitro. Expression increased in BFU-E cultures that were exposed to EPO when compared to controls.

Expression of the mouse 25584 ortholog was shown to be most highly expressed in lineage negative progenitors and erythroid TER119+ cells.

25584 is involved in the hydrolysis of N-acylated or N-acetylated amino acids (except L-aspartate). An association between reduced levels of aminoacylase 1 and increased proliferation of lung cancer cells has been observed (J. Biol. Chem. 1993 Aug. 15; 268(23): 17010-7). The function of aminoacylase 1 leads to decreased proliferation (potentially through degradation of N-acyl amino acids). Inhibition of this enzyme would lead to increased proliferation of erythroid progenitors

Due to 25584 mRNA expression, along with its functional role, modulators of 25584 would be useful in the treatment of hematological disorders. 25584 polypeptides of the present invention would be useful in screening for modulators of 25584 activity.

Gene ID 27824

The human 27824 sequence (SEQ ID NO:55), known also as that ubiquitin carboxyl-terminal hydrolase isozyme L5, is approximately 1728 nucleotides long including untranslated regions. The coding sequence, located at about nucleic acid 132 to 1121 of SEQ ID NO:55, encodes a 329 amino acid protein (SEQ ID NO:56).

As assessed by TaqMan analysis, 27824 mRNA had restricted expression. 27824 mRNA had very high levels of expression in erythroid cells, progenitor cells and brain, and lower levels of expression in other tissues and organs. In hematopoietic cells, 27824 was expressed in a very restricted manner, with high expression in pooled Day 10 erythroid cultures and K562 erythroleukemia cells. Lower expression was observed in CD34+ progenitors (which also contain erythroid progenitors). Upon differentiation of erythroid cells in vitro, the expression of 27824 mRNA increased, peaking at day 6, which corresponds to the highest erythroid specific proliferation potential of the cells. 27824 mRNA expression was also at higher levels in GPA low (immature cells) when compared to the more mature GPA high cells. These data suggest that this enzyme has a role in erythroid progenitors.

Mouse hematopoietic cells express the ortholog of 27824. The highest expression levels of mouse 27824 mRNA were observed in TER119+ erythroid cells. Lineage negative cells, which contain the erythroid progenitors, also expressed 27824 mRNA.

Ubiquitination of the EPO receptor is required for maximal signaling (J Biol. Chem. 2003 Jul. 18; 278(29):26851-61.) This enzyme, namely ubiquitin carboxyl-terminal hydrolase isozyme L5, is involved in de-ubiquitination. Ubiquitin carboxyl-terminal hydrolase isozyme L5 negatively regulates the EPO receptor by de-ubiquitinating it. As ubiquitination is required for signaling through the EPO receptor, inhibition of this enzyme would lead to more signaling through the EPO receptor and ultimately lead to increased erythroid cell numbers.

Due to 27824 mRNA expression, along with its functional role, modulators of 27824 would be useful in the treatment of hematological disorders. 27824 polypeptides of the present invention would be useful in screening for modulators of 27824 activity.

Gene ID 28469

The human 28469 sequence (SEQ ID NO:57), known also as O-sialoglycoprotein endopeptidase, is approximately 1008 nucleotides long. The coding sequence, located at about nucleic acid 1 to 1008 of SEQ ID NO:57, encodes a 335 amino acid protein (SEQ ID NO:58).

As assessed by TaqMan analysis, 28469 mRNA was expressed at the highest levels in HUVECs, brain, megakaryocytes and erythroid cells. In hematopoietic cells, the highest expression levels of 28469 mRNA was observed in Day 10 pooled erythroid samples, Day 10 pooled megakaryocyte cultures, fetal liver and K652 cells. 28469 mRNA expression is detectable but lower in T-cells, myeloid cells and CD34 progenitors. 28469 mRNA expression levels in erythroid cells were higher in GPA low progenitors than in the more mature GPA high cells. 28469 mRNA expression increased rapidly during erythroid in vitro differentiation, peaking between 48 hrs and 6 days in culture, in parallel with the maximal proliferative potential of the cells. In vitro megakaryocytic cultures also showed 28469 mRNA expression but it was not regulated with differentiation.

The mouse ortholog of 28469 was expressed in mouse hematopoietic cells, with the highest levels of expression in the erythroid TER119+ cells. 28469 mRNA expression was also present at significant levels in the lineage negative progenitor fraction.

O-sialoglycoprotein endopeptidase has recently been cloned and is described as a homologue of gcp, a Pasteurella haemolytica A1 glycoprotease, with 29.7% identity (Gene. 2002 Feb. 20; 285(1-2):101-8). The best-characterized substrate of Pasteurella glycoprotease is glycophorin A. Several other O-sialoglycoprotein substrates are human leucocyte antigens including CD43, CD45, CD44 and P-selectin ligand. This enzyme also cleaves CD34, a glycoprotein on the surface of human hematopoietic stem cells. The adhesive characteristics of hematopoietic stem and progenitor cells may partly regulate their proliferation and differentiation (Ann N Y Acad Sci. 2001 Jun.; 938:196-206; discussion 206-7.) Maintenance of CD43 adhesion is associated with proliferation of TF-1 cells (Biochem Biophys Res Commun. 2001 Oct. 19; 288(1):80-6.) Inhibition of O-sialoglycoprotein endopeptidase function would lead to maintenance of erythroid antigens important for survival and thus lead to increased numbers of erythroid cells.

Due to 28469 mRNA expression, along with its functional role, modulators of 28469 would be useful in the treatment of hematological disorders. 28469 polypeptides of the present invention would be useful in screening for modulators of 28469 activity.

Gene ID 38947

The human 38947 sequence (SEQ ID NO:59), known also as protein phosphatase methylesterase-1, is approximately 2484 nucleotides long including untranslated regions. The coding sequence, located at about nucleic acid 100 to 1260 of SEQ ID NO:59, encodes a 386 amino acid protein (SEQ ID NO:60).

As assessed by TaqMan analysis, 38947 mRNA was expressed almost exclusively in hematopoietic cells with the highest expression in GPA low erythroid cells and in Day 10 in vitro differentiated erythroid cultures. 38947 mRNA was also highly expressed in the K562 erythroid cell line. Expression of 38947 mRNA was observed to increase significantly with erythroid differentiation in vitro. Furthermore, 38947 mRNA expression is higher in GPA low erythroid cells when compared to the more mature GPA high cells. Finally, 38947 mRNA expression was observed in lineage negative and TER 119+ cells that correspond to erythroid progenitors and mature cells.

38947 or protein phosphatase methylesterase-1 is known to demethylate and inactivate the protein phosphatase 2A catalytic subunit (J. Biol. Chem., 274:14382-14391). It is the current invention that inhibition of 38947 will result in increased proliferation of erythroid progenitors and therefore would be useful for the treatment of hematological disorders. This invention is based at least in part on the studies first described herein, which demonstrate the differential and exclusive expression of 38947 in erythroid cells and upon previous studies, which demonstrate that this enzyme inhibits PP2A.

Due to 38947 mRNA expression, along with its functional role, modulators of 38947 would be useful in the treatment of hematological disorders. 38947 polypeptides of the present invention would be useful in screening for modulators of 38947 activity.

Gene ID 53003

The human 53003 sequence (SEQ ID NO:61), known also as acyl-CoA synthetase MACS 1, is approximately 2074 nucleotides long including untranslated regions. The coding sequence, located at about nucleic acid 79 to 1812 of SEQ ID NO:61, encodes a 577 amino acid protein (SEQ ID NO:62).

As assessed by TaqMan analysis, 53003 mRNA expression was very restricted, detectable only in erythroid cells, ovary and pancreas. 53003 mRNA expression in hematopoietic cells was very restricted to Day 10 pooled differentiating erythroid cells. 53003 mRNA expression was also detected in CD34+ progenitors. During erythroid differentiation, 53003 mRNA expression was strongly up regulated at day 6 in a manner that very closely resembles EPO receptor expression. It was also down regulated in GPA low cells when compared to GPA high cells. Furthermore, 53003 mRNA expression was increased in BFU-E cultures with EPO compared to those without EPO.

Expression of the 53003 mouse ortholog was detectable in mouse hematopoeitic cells, particularly in lineage negative progenitors.

53003 is a medium chain acyl-CoA synthetase, also called butyryl-CoA synthetase, which acts on acids from C4 to C11 and on the corresponding 3-hydroxy- and 2,3- or 3,4-unsaturated acids with preference for octanoate as its substrate (J Biol. Chem. 2001 Sep. 21; 276(38):35961-6. Epub 2001 Jul. 24). The substrates of MACS1 would lead to differentiation of erythroid cells and its inhibition would lead to increased proliferation of CFU-Es and generation of more erythroid cells.

Due to 53003 mRNA expression in erythroid cells, along with its functional role, modulators of 53003 would be useful in the treatment of hematological disorders. 53003 polypeptides of the present invention would be useful in screening for modulators of 53003 activity.

Gene ID 965

The human 965 sequence (SEQ ID NO:63), known also as protein-tyrosine phosphatase delta (R-PTP-delta), is approximately 6263 nucleotides long including untranslated regions. The coding sequence, located at about nucleic acids 154 to 5892 of SEQ ID NO:63, encodes a 1912 amino acid protein (SEQ ID NO:64).

As determined by TaqMan analysis, 965 mRNA was expressed at the highest levels in brain and expressed at low levels in most tissues and organs. In hematopoieitic cells, 965 mRNA was expressed at the highest levels in CD34 progenitor cells, but not in differentiated hematopoieitic cells. In cultures differentiated to myeloid cells, erythroid cells and megakaryocytes, 965 mRNA expression decreases after 24 and 48 hours in culture, indicating that its key role is in CD34 progenitor cells.

Expression of the mouse ortholog of 965 was detected in lineage negative fractions of the bone marrow corresponding to the progenitor cell population. It was also detected in bone marrow white blood cells, TER119+ erythroid cells and in Gr-1+ neutrophils.

PTP-delta is selectively decreased in primary hepatomas and hepatoma cell lines, suggesting that it is a tumor suppressor. Inhibition of PTP-delta function with an inhibitor would lead to increased proliferation of CD34 progenitor cells and a subsequent increase in erythropoiesis, myelopoiesis and megakaryopoiesis.

Due to 965 mRNA expression; along with its functional role, modulators of 965 would be useful in the treatment of hematological disorders. 965 polypeptides of the present invention would be useful in screening for modulators of 965 activity.

Gene ID 56639

The human 56639 sequence (SEQ ID NO:65), known also as proprotein convertase 4 (PC4), is approximately 2669 nucleotides long including untranslated regions. The coding sequence, located at about nucleic acids 61 to 2328 of SEQ ID NO:65, encodes a 755 amino acid protein (SEQ ID NO:66).

As assessed by TaqMan analysis, 56639 mRNA was expressed at the highest levels in erythroid cells, brain, pituitary gland and kidney. In hematopoieitic cells, 56639 mRNA was expressed at very high levels in pooled Day 10 differentiating erythroid cells, in K562 (erythroleukemia cell line) and in CD34 cells. In erythroid cells, 56639 mRNA expression increased in BFU-E cultures in the presence of EPO. 56639 mRNA expression was detected in GPA low cells at higher levels than in GPA high samples. Low expression of 56639 mRNA was detected in megakaryocytic and neutrophil cultures.

Expression of the mouse ortholog of 56639 was detected in lineage negative fractions of the bone marrow corresponding to the progenitor cell population.

Proprotein convertases are calcium-dependent serine proteases related to bacterial subtilisins and to yeast kexin. These enzymes process precursor proteins to their active forms by selective cleavage of the polypeptide at sites following paired basic amino acids. In mammals, this family comprises PC1, PC2, PC4, PC5, furin (FUR), and PACE4. Substrates for these enzymes range from prohormones to precursors for growth factors to cell surface receptors and viral surface glycoproteins. This enzyme is highly expressed in proliferating erythroid progenitors. It may function in growth factor or other protein degradation to control the proliferation rate of these cells or function to process proteins in the apoptotic pathway. Inhibiting 56639 will lead to expansion of erythroid cells.

Due to 56639 mRNA expression, along with its functional role, modulators of 56639 would be useful in the treatment of hematological disorders. 56639 polypeptides of the present invention would be useful in screening for modulators of 56639 activity.

Gene ID 9661

The human 9661 sequence (SEQ ID NO:67), known also as cyclin dependent kinase associated phosphatase (KAP), is approximately 844 nucleotides long including untranslated regions. The coding sequence, located at about nucleic acids 52 to 690 of SEQ ID NO:67, encodes a 212 amino acid protein (SEQ ID NO:68).

As assessed by TaqMan analysis, 9661 mRNA had restricted expression in hematopoietic cells. In vitro differentiated Day 10 erythroid cultures showed the highest expression levels of 9661 mRNA. In erythroid cells, 9661 mRNA expression increased significantly with erythroid cell differentiation in vitro and was expressed at higher levels in GPA low erythroid cells when compared to the more mature GPA high cells. There was also some expression in skeletal muscle.

The mouse ortholog of 9661 was expressed in lineage negative cells and TER119+ cells corresponding to erythroid progenitors and mature cells.

9661 may play a role in cell cycle regulation. It is a dual specificity phosphatase which is active toward substrates containing either phosphotyrosine or phosphoserine residues. 9661 interacts with cyclin-dependent kinases such as CDC2, CDK2 and CDK3, but does not interact with CDK4. It is believed that this phosphatase is a negative regulator of proliferation. This is based on observations that certain mutations no longer permit it to interact with Cdk2 in hepatocellular carcinoma (Cancer Res. 2000 Sep. 1; 60(17):4697-700; Biochem Biophys Res Commun. 2003 May 30; 305(2):311-4). Inhibition of this phosphatase will lead to increased proliferation of erythroid precursors.

Due to 9661 mRNA expression, along with its functional role, modulators of 9661 would be useful in the treatment of hematological disorders. 9661 polypeptides of the present invention would be useful in screening for modulators of 9661 activity.

Gene ID 16052

The human 16052 sequence (SEQ ID NO:69), known also as protein-tyrosine phosphatase, non-receptor type 22 or LYP1, is approximately 3058 nucleotides long including untranslated regions. The coding sequence, located at about nucleic acid 42 to 2468 of SEQ ID NO:69, encodes a 808 amino acid protein (SEQ ID NO:70).

As assessed by TaqMan analysis, 16052 mRNA was expressed at the highest levels in developing CD11b−, CD14+, CD15+ neutrophils and CD11b+, CD 14+, CD15+ cells. It was also seen in neutrophil differentiation cultures in vitro. 16052 mRNA expression decreased as erythroid progenitors and megakaryocytes differentiate from CD34 cells in vitro. 16052 mRNA was detected at lower levels in T-cells, B-cells and progenitors. 16052 mRNA was also expressed at low levels in tissues and organs but can be detected in lymphoid tissue, spleen, and skin.

The mouse ortholog of 16052 was also expressed in hematopoietic cells, with the highest levels in bone marrow white blood cells, and in the subfractions of Gr1+ neutrophils and Ter119+ erythroid cells. 16052 mRNA expression increased with increasing maturity from bone marrow lineage negative cells to Gr1+ neutrophils, with higher levels seen in the Gr1 medium than in the Gr1 low fraction.

16052 was cloned and described as hematopoietic cell protein-tyrosine phosphatase 70Z-PEP or lymphoid phosphatase. It has been described as “predominantly expressed in lymphoid tissues and cells. Isoform 1 is expressed in thymocytes and both mature B and T cells” (Blood. 1999 Mar. 15; 93(6):2013-24). It has been shown to function as a negative regulator of T-cell activation (Exp Hematol. 2002 Mar.; 30(3):237-44). This phosphatase has been shown to negatively regulate proliferative signals (like the aberrant BCR-ABL observed in chronic myeloid leukemia) suggesting that it may serve as a negative regulator of normal pro-proliferative signals also (J Biol. Chem. 2003 Jul. 25; 278(30):27413-20. Epub 2003 May 21). Inhibition of the function of this enzyme in myeloid cells will lead to proliferation and an increase in the number of neutrophils.

Due to 16052 mRNA expression, along with its functional role, modulators of 16052 would be useful in the treatment of hematological disorders. 16052 polypeptides of the present invention would be useful in screening for modulators of 16052 activity.

Gene ID 1521

The human 1521 sequence (SEQ ID NO:71), known also as glycogen synthase kinase 3 beta (GSK3beta), is approximately 1389 nucleotides long including untranslated regions. The coding sequence, located at about nucleic acids 40 to 1302 of SEQ ID NO:71, encodes a 420 amino acid protein (SEQ ID NO:72).

As assessed by TaqMan analysis, 1521 mRNA was expressed at the highest levels in differentiated Day 10 erythroid in vitro cultures. It was also expressed in the erythroid cell line K562 and in GPA low erythroid cells. 1521 mRNA was expressed at lower levels in CD34+ progenitors and was detectable in CD3+ T-cells and CD19+ B-cells. In erythroid cells, 1521 mRNA expression increased during erythroid differentiation in vitro, increasing from 24 hours in culture and peaking at 6 days, corresponding to the EPO receptor. 1521 mRNA expression was also at higher levels in GPA low erythroid cells when compared to the more mature GPA high expressing cells. In an organ recital, 1521 mRNA expression was quite restricted and was at the highest levels in erythroid cells with lower expression in progenitor cells. There was also some expression in skeletal muscle and brain.

The mouse ortholog of 1521 was expressed in lineage negative cells and TER119+ cells corresponding to erythroid progenitors and mature cells. It was also expressed in the Gr1 fraction of cells.

GSK3beta has been described as contributing to pro-apoptotic signaling (J Biol. Chem. 2001 Oct. 5; 276(40):37436-42. Epub 2001 Aug. 08). It has also been described as a being downstream of AKT and negatively regulated by it (J. Neurochem. 2000 Dec.; 75(6):2401-8.) It has also been described as a negative regulator of wnt signaling (Proc Natl Acad Sci USA. 1995 Aug. 29; 92(18):8498-502.) Wnt signaling from stromal cells to erythroid cells leads to an increased formation of CFU-Es. WNT5A and GSK3beta are expressed in CD34+ cells (Blood. 1998 Nov. 1; 92(9):3189-202.) Inhibition of this enzyme in CD34+ cells and later erythroid progenitors will lead to increased signaling through the Wnt pathways and AKT, leading to increased erythroid cell proliferation.

Due to 1521 mRNA expression, along with its functional role, modulators of 1521 would be useful in the treatment of hematological disorders. 1521 polypeptides of the present invention would be useful in screening for modulators of 1521 activity.

Gene ID 6662

The human 6662 sequence (SEQ ID NO:73), known also as protein phosphatase 2C gamma isoform (PP2Cgamma), is approximately 1932 nucleotides long including untranslated regions. The coding sequence, located at about nucleic acids 25 to 1665 of SEQ ID NO:73, encodes a 546 amino acid protein (SEQ ID NO:74).

As assessed by TaqMan analysis, 6662 mRNA had a restricted expression profile in hematopoietic cells, with highest expression levels detected in in vitro differentiated Day 10 erythroid cultures. 6662 mRNA was also expressed in the erythroid cell line K562 and in GPA low erythroid cells. 6662 mRNA was expressed at lower levels in CD34+ progenitors and was detectable in CD3+ T-cells and CD 19+ B-cells. In erythroid cells, 6662 mRNA expression increased significantly with erythroid differentiation in vitro, increasing from 24 hours in culture onwards. It was also at higher levels in GPA low erythroid cells when compared to the more mature GPA high expressing cells. In an organ recital, 6662 mRNA expression was quite restricted and was at the highest levels in erythroid cells. There was also some expression in skeletal muscle.

The mouse ortholog of 6662 was expressed in lineage negative cells and TER119+ cells corresponding to the erythroid progenitors and mature cells. 6662 mRNA expression increased in the mouse ‘EPO-on’ model (where animals are treated with EPO and the lineage negative population is studied). 6662 mRNA expression was at higher levels in the samples treated with EPO than in the controls. The levels of expression also increased in the group of “anemia by phlebotomy” mouse model. In both of these models, the increase in expression of this phosphatase correlates with an increase in expression of the EPO receptor.

PP2C has been described as a negative regulator of MAPK kinase signaling (J Biol. Chem. 2003 May 23; 278(21):18945-52. Epub 2003 Mar. 19.; EMBO J. 1998 Aug. 17; 17(16):4744-52). The alpha isoform of PP2C has been described in p53 activation (J Biol. Chem. 2003 Apr. 18; 278(16):14299-305. Epub 2003 Jan. 03). p53 deficient cells have an increased proliferative potential compared to wild type controls (Exp Biol Med (Maywood). 2002 Jul.; 227(7):474-9). PP2C phosphatases appear to have a role in regulation of proliferation. PP2C family members have been described as having a role in splicing and there is evidence to suggest that the gamma isoform has a role in this process (Genes Dev. 1999 Jan. 1;13(1):87-97). PP2C gamma, like the other PP2C isoforms, may have a role in negative regulation of proliferative signaling. Inhibition of PP2Cgamma will lead to increased proliferation of erythroid progenitors.

Due to 6662 mRNA expression, along with its functional role, modulators of 6662 would be useful in the treatment of hematological disorders. 6662 polypeptides of the present invention would be useful in screening for modulators of 6662 activity.

Gene ID 13913

The human 13913 sequence (SEQ ID NO:75), known also as metalloprotease 1, is approximately 3432 nucleotides long including untranslated regions. The coding sequence, located at about nucleic acids 14 to 3130 of SEQ ID NO:75, encodes a 1038 amino acid protein (SEQ ID NO:76).

As assessed by TaqMan analysis, 13913 mRNA expression was restricted to hematopoietic cells, with the highest expression levels shown in in vitro differentiated day 10 erythroid cultures. 13913 mRNA was also expressed in the erythroid cell line K562 and GPA low erythroid cells. 13913 mRNA was additionally expressed at lower levels in CD34+ progenitor cells and was detectable in neutrophils, monocytes, T-cells (CD3+) and B-cells (CD19+). Further TaqMan experiments conducted on an erythroid tissue panel demonstrated that 13913 mRNA expression increased significantly with erythroid differentiation in vitro. Expression increased from 24 hours in culture onwards, peaking at day 6 in culture. 13913 mRNA was also expressed at higher levels in GPA low erythroid cells compared to the more mature GPA high expressing cells and was increased in BFU-E cultures in response to EPO.

The mouse ortholog of 13913 was expressed most highly in TER119+ erythroid cells and lineage negative progenitor cells (which includes erythroid progenitors). It was expressed at lower levels in myeloid cells: GR1+ neutrophils and mononuclear cells. In a mouse model where cells are treated with EPO, the expression level of this protease increases in EPO treated lineage negative cells. This parallels an observed increase in expression of the EPO receptor.

The catalytic domain in this human metalloprotease 1 is conserved in the members of pitrilysin family such as insulin-degrading enzyme (DNA Cell Biol. 1999 May;18(5):369-80). 13913 is expressed in proliferating erythroid progenitor cells and may therefore function in growth factor or other protein degradation pathways to control the proliferation rate of erythroid cells. Inhibition of 13913 may therefore lead to expansion of erythroid cells.

Due to 13913 mRNA expression, along with its functional role, modulators of 13913 would be useful in the treatment of hematological disorders. 13913 polypeptides of the present invention would be useful in screening for modulators of 13913 activity.

Gene ID 12405

The human 12405 sequence (SEQ ID NO:77), known also as serine palmitoyltransferase, is approximately 2026 nucleotides long including untranslated regions. The coding sequence, located at about nucleic acids 49 to 1737 of SEQ ID NO:77, encodes a 562 amino acid protein (SEQ ID NO:78).

As assessed by TaqMan analysis, 12405 mRNA had a restricted expression profile in hematopoietic cells, with highest expression levels shown in in vitro differentiated Day 10 erythroid cultures. 12405 mRNA was also expressed in the erythroid cell line K562 and in GPA low erythroid cells. 12405 mRNA was additionally expressed at lower levels in CD34+ progenitors and was detectable in neutrophils, monocytes, T-cells (CD3+) and B-cells (CD19+). Further TaqMan experiments conducted on an erythroid tissue panel demonstrated that 12405 mRNA expression increased significantly with erythroid differentiation in vitro. Expression increased from 24 hours in culture onwards, peaking at day 6 in culture. 12405 mRNA expression was also shown to be at higher levels in GPA low erythroid cells when compared to the more mature GPA high expressing cells and was increased in BFU-E cultures in response to EPO.

The mouse ortholog of 12405 was expressed at equal levels in lineage negative cells and TER119+ cells corresponding to the erythroid progenitors and mature cells. It was also expressed in the neutrophils (Gr1+).

12405, also known as serine palmitoyltransferase, is the rate limiting enzyme in the production of ceramide. Ceramide has been described as a proapoptotic factor, inducing cell cycle arrest and differentiation and it has been linked to apoptosis in erythroid cells (Cancer Lett. 2003 Apr. 25;193(2):149-54; Exp Hematol. 1999 Jul. 27(7):1133-8). Therefore, inhibition of serine palmitoyl transferase may reduce the production of ceramide, and increase the survival and proliferation of erythroid progenitor cells.

Due to 12405 mRNA expression, along with its functional role, modulators of 12405 would be useful in the treatment of hematological disorders. 12405 polypeptides of the present invention would be useful in screening for modulators of 12405 activity.

Gene ID 5014

The human 5014 sequence (SEQ ID NO:79), known also as protein phosphatase 2C alpha isoform (PP2Calpha), is approximately 2346 nucleotides long including untranslated regions. The coding sequence, located at about nucleic acid 358 to 1506 of SEQ ID NO:79, encodes a 382 amino acid protein (SEQ ID NO:80).

As assessed by TaqMan analysis, 5014 mRNA expression was mainly restricted to hematopoietic cells, being at the highest levels in in vitro differentiated Day 10 erythroid cultures. Lower levels of expression were observed in skeletal muscle and in brain samples. 5014 mRNA was also expressed in the erythroid cell line K562, in GPA low erythroid cells and in fetal liver. It was expressed at lower levels in CD34+ progenitor cells and it was detectable in T-cells (CD3+) and B-cells (CD19+). Additional TaqMan analyses using an erythroid panel, demonstrated that 5014 mRNA expression increased significantly with erythroid differentiation in vitro. 5014 mRNA expression increased from 24 hours in culture onwards, and expression also increased in BFU-E cultures in the presence of EPO. 5014 mRNA expression was observed in both GPA low erythroid cells and in GPA high expressing cells.

The mouse ortholog of 5014 showed expression in lineage negative cells and TER 119+ cells corresponding to the erythroid progenitors and mature cells. It was also expressed in mononuclear cells and GR1+ive neutrophils.

5014 is also known as protein phosphatase 2C alpha isoform (PP2Calpha). PP2C has been described as a negative regulator of MAPK kinase signaling (J. Biol. Chem. 2003 May 23;278(21): 18945-52. Epub 2003 Mar. 19; and EMBO J. 1998 Aug. 17;17(16):4744-52). The alpha isoform of PP2C has been described in p53 activation (J. Biol. Chem. 2003 Apr. 18;278(16):14299-305. Epub 2003 Jan. 03). p53 deficient cells have an increased proliferative potential compared to wild type controls (Exp Biol Med (Maywood). 2002 Jul.;227(7):474-9). Therefore, PP2C phosphatases appear to have a role in regulation of proliferation. PP2Calpha may also have a role in the negative regulation of proliferative signaling. Inhibition of PP2Cgamma may therefore lead to increased proliferation of erythroid progenitor cells.

Due to 5014 mRNA expression in erythroid cells, along with its functional role, modulators of 5014 would be useful in the treatment of hematological disorders. 5014 polypeptides of the present invention would be useful in screening for modulators of 5014 activity.

Various aspects of the invention are described in further detail in the following subsections:

I. Screening Assays:

The invention provides a method (also referred to herein as a “screening assay”) for identifying modulators, i.e., candidate or test compounds or agents (e.g., peptides, peptidomimetics, small molecules (organic or inorganic) or other drugs) which bind to 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 proteins, have a stimulatory or inhibitory effect on, for example, 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 expression or 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 activity, or have a stimulatory or inhibitory effect on, for example, the expression or activity of a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 substrate. Compounds identified using the assays described herein may be useful for treating hematological disorders.

These assays are designed to identify compounds that bind to a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein, bind to other intracellular or extracellular proteins that interact with a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein, and interfere with the interaction of the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein with other intercellular or extracellular proteins. For example, in the case of the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein, which is a transmembrane receptor-type protein, such techniques can identify ligands for such a receptor. A 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein ligand or substrate can, for example, be used to at least one symptom of a hematological disorder. Such compounds may include, but are not limited to peptides, antibodies, or small organic or inorganic compounds. Such compounds may also include other cellular proteins.

Compounds identified via assays such as those described herein may be useful, for example, for treating hematological disorders. In instances whereby a hematological disorder condition results from an overall lower level of 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 gene expression and/or 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein in a cell or tissue, compounds that interact with the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein may include compounds which accentuate or amplify the activity of the bound 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein. Such compounds would bring about an effective increase in the level of 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein activity, thus ameliorating symptoms.

In other instances, mutations within the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 gene may cause aberrant types or excessive amounts of 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 proteins to be made which have a deleterious effect that leads to a hematological disorder. Similarly, physiological conditions may cause an excessive increase in 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 gene expression leading hematological disorders. In such cases, compounds that bind to a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein may be identified that inhibit the activity of the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein. Assays for testing the effectiveness of compounds identified by techniques such as those described in this section are discussed herein.

In one embodiment, the invention provides assays for screening candidate or test compounds which are substrates of a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein or polypeptide or biologically active portion thereof. In another embodiment, the invention provides assays for screening candidate or test compounds which bind to or modulate the activity of a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein or polypeptide or biologically active portion thereof. The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990) Proc. Natl. Acad. Sci. 87:6378-6382); (Felici (1991) J. Mol. Biol. 222:301-310); (Ladner supra.).

In one embodiment, an assay is a cell-based assay in which a cell which expresses a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein or biologically active portion thereof is contacted with a test compound and the ability of the test compound to modulate 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 activity is determined. Determining the ability of the test compound to modulate 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 activity can be accomplished by monitoring, for example, intracellular calcium, IP₃, cAMP, or diacylglycerol concentration, the phosphorylation profile of intracellular proteins, cell proliferation and/or migration, gene expression of, for example, cell surface adhesion molecules or genes associated with hematopoeisis, or the activity of a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014-regulated transcription factor. The cell can be of mammalian origin, e.g., a neural cell. In one embodiment, compounds that interact with a receptor domain can be screened for their ability to function as ligands, i.e., to bind to the receptor and modulate a signal transduction pathway. Identification of ligands, and measuring the activity of the ligand-receptor complex, leads to the identification of modulators (e.g., antagonists) of this interaction. Such modulators may be useful in the treatment of hematological disorders.

The ability of the test compound to modulate 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 binding to a substrate or to bind to 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 can also be determined. Determining the ability of the test compound to modulate 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 binding to a substrate can be accomplished, for example, by coupling the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 substrate with a radioisotope or enzymatic label such that binding of the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 substrate to 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 can be determined by detecting the labeled 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 substrate in a complex. 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 could also be coupled with a radioisotope or enzymatic label to monitor the ability of a test compound to modulate 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 binding to a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 substrate in a complex. Determining the ability of the test compound to bind 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 can be accomplished, for example, by coupling the compound with a radioisotope or enzymatic label such that binding of the compound to 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 can be determined by detecting the labeled 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 compound in a complex. For example, compounds (e.g., 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 ligands or substrates) can be labeled with ¹²⁵I, ³⁵S, ¹⁴C, or ³H, either directly or indirectly, and the radioisotope detected by direct counting of radioemmission or by scintillation counting. Compounds can further be enzymatically labeled with, for example, horseradish peroxidase, alkaline phosphatase, or luciferase, and the enzymatic label detected by determination of conversion of an appropriate substrate to product.

It is also within the scope of this invention to determine the ability of a compound (e.g., a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 ligand or substrate) to interact with 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 without the labeling of any of the interactants. For example, a microphysiometer can be used to detect the interaction of a compound with 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 without the labeling of either the compound or the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 (McConnell, H. M. et al. (1992) Science 257:1906-1912. As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014.

In another embodiment, an assay is a cell-based assay comprising contacting a cell expressing a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 target molecule (e.g., a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 substrate) with a test compound and determining the ability of the test compound to modulate (e.g., stimulate or inhibit) the activity of the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 target molecule. Determining the ability of the test compound to modulate the activity of a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 target molecule can be accomplished, for example, by determining the ability of the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein to bind to or interact with the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 target molecule.

Determining the ability of the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein or a biologically active fragment thereof, to bind to or interact with a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 target molecule can be accomplished by one of the methods described above for determining direct binding. In a preferred embodiment, determining the ability of the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein to bind to or interact with a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 target molecule can be accomplished by determining the activity of the target molecule. For example, the activity of the target molecule can be determined by detecting induction of a cellular second messenger of the target (i.e., intracellular Ca²⁺, diacylglycerol, IP₃, cAMP), detecting catalytic/enzymatic activity of the target on an appropriate substrate, detecting the induction of a reporter gene (comprising a target-responsive regulatory element operatively linked to a nucleic acid encoding a detectable marker, e.g., luciferase), or detecting a target-regulated cellular response (e.g., gene expression).

In yet another embodiment, an assay of the present invention is a cell-free assay in which a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein or biologically active portion thereof, is contacted with a test compound and the ability of the test compound to bind to the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein or biologically active portion thereof is determined. Preferred biologically active portions of the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 proteins to be used in assays of the present invention include fragments which participate in interactions with non-9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 molecules, e.g., fragments with high surface probability scores. Binding of the test compound to the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein can be determined either directly or indirectly as described above. In a preferred embodiment, the assay includes contacting the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein or biologically active portion thereof with a known compound which binds 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 to form an assay mixture, contacting the assay mixture with a test compound, and determining the ability of the test compound to interact with a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein, wherein determining the ability of the test compound to interact with a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein comprises determining the ability of the test compound to preferentially bind to 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 or biologically active portion thereof as compared to the known compound. Compounds that modulate the interaction of 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 with a known target protein may be useful in regulating the activity of a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein, especially a mutant 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein.

In another embodiment, the assay is a cell-free assay in which a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein or biologically active portion thereof is contacted with a test compound and the ability of the test compound to modulate (e.g., stimulate or inhibit) the activity of the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein or biologically active portion thereof is determined. Determining the ability of the test compound to modulate the activity of a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein can be accomplished, for example, by determining the ability of the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein to bind to a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 target molecule by one of the methods described above for determining direct binding. Determining the ability of the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein to bind to a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 target molecule can also be accomplished using a technology such as real-time Biomolecular Interaction Analysis (BIA) (Sjolander, S. and Urbaniczky, C. (1991) Anal. Chem. 63:2338-2345 and Szabo et al. (1995) Curr. Opin. Struct. Biol. 5:699-705). As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules.

In another embodiment, determining the ability of the test compound to modulate the activity of a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein can be accomplished by determining the ability of the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein to further modulate the activity of a downstream effector of a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 target molecule. For example, the activity of the effector molecule on an appropriate target can be determined or the binding of the effector to an appropriate target can be determined as previously described.

In yet another embodiment, the cell-free assay involves contacting a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein or biologically active portion thereof with a known compound which binds the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein to form an assay mixture, contacting the assay mixture with a test compound, and determining the ability of the test compound to interact with the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein, wherein determining the ability of the test compound to interact with the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein comprises determining the ability of the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein to preferentially bind to or modulate the activity of a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 target molecule.

In more than one embodiment of the above assay methods of the present invention, it may be desirable to immobilize either 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 or its target molecule to facilitate separation of complexed from uncomplexed forms of one or both of the proteins, as well as to accommodate automation of the assay. Binding of a test compound to a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein, or interaction of a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein with a target molecule in the presence and absence of a candidate compound, can be accomplished in any vessel suitable for containing the reactants. Examples of such vessels include microtitre plates, test tubes, and micro-centrifuge tubes. In one embodiment, a fusion protein can be provided which adds a domain that allows one or both of the proteins to be bound to a matrix. For example, glutathione-S-transferase/9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 fusion proteins or glutathione-5-transferase/target fusion proteins can be adsorbed onto glutathione sepharose beads (Sigma Chemical, St. Louis, Mo.) or glutathione derivatized microtitre plates, which are then combined with the test compound or the test compound and either the non-adsorbed target protein or 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein, and the mixture incubated under conditions conducive to complex formation (e.g., at physiological conditions for salt and pH). Following incubation, the beads or microtitre plate wells are washed to remove any unbound components, the matrix immobilized in the case of beads, complex determined either directly or indirectly, for example, as described above. Alternatively, the complexes can be dissociated from the matrix, and the level of 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 binding or activity determined using standard techniques.

Other techniques for immobilizing proteins on matrices can also be used in the screening assays of the invention. For example, either a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein or a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 target molecule can be immobilized utilizing conjugation of biotin and streptavidin. Biotinylated 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein or target molecules can be prepared from biotin-NHS(N-hydroxy-succinimide) using techniques known in the art (e.g., biotinylation kit, Pierce Chemicals, Rockford, Ill.), and immobilized in the wells of streptavidin-coated 96 well plates (Pierce Chemical). Alternatively, antibodies reactive with 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein or target molecules but which do not interfere with binding of the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein to its target molecule can be derivatized to the wells of the plate, and unbound target or 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein trapped in the wells by antibody conjugation. Methods for detecting such complexes, in addition to those described above for the GST-immobilized complexes, include immunodetection of complexes using antibodies reactive with the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein or target molecule, as well as enzyme-linked assays which rely on detecting an enzymatic activity associated with the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein or target molecule.

In another embodiment, modulators of 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 expression are identified in a method wherein a cell is contacted with a candidate compound and the expression of 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 mRNA or protein in the cell is determined. The level of expression of 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 mRNA or protein in the presence of the candidate compound is compared to the level of expression of 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 mRNA or protein in the absence of the candidate compound. The candidate compound can then be identified as a modulator of 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 expression based on this comparison. For example, when expression of 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 mRNA or protein is greater (statistically significantly greater) in the presence of the candidate compound than in its absence, the candidate compound is identified as a stimulator of 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 mRNA or protein expression. Alternatively, when expression of 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 mRNA or protein is less (statistically significantly less) in the presence of the candidate compound than in its absence, the candidate compound is identified as an inhibitor of 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 mRNA or protein expression. The level of 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 mRNA or protein expression in the cells can be determined by methods described herein for detecting 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 mRNA or protein.

In yet another aspect of the invention, the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 proteins can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other proteins, which bind to or interact with 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 (“ 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014-binding proteins” or “9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014-bp”) and are involved in 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 activity. Such 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014-binding proteins are also likely to be involved in the propagation of signals by the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 proteins or 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 targets as, for example, downstream elements of a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014-mediated signaling pathway. Alternatively, such 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014-binding proteins are likely to be 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 inhibitors.

The two-hybrid system is based on the modular nature of most transcription factors, which consist of separable DNA-binding and activation domains. Briefly, the assay utilizes two different DNA constructs. In one construct, the gene that codes for a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein.

In another aspect, the invention pertains to a combination of two or more of the assays described herein. For example, a modulating agent can be identified using a cell-based or a cell free assay, and the ability of the agent to modulate the activity of a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein can be confirmed in vivo, e.g., in an animal such as an animal model for hematological disorders, as described herein.

This invention further pertains to novel agents identified by the above-described screening assays. Accordingly, it is within the scope of this invention to further use an agent identified as described herein in an appropriate animal model. For example, an agent identified as described herein (e.g., a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 modulating agent, an antisense 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 nucleic acid molecule, a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014-specific antibody, or a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014-binding partner) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

Any of the compounds, including but not limited to compounds such as those identified in the foregoing assay systems, may be tested for the ability to treat hematological disorders. Cell-based and animal model-based assays for the identification of compounds exhibiting such an ability to at least one symptom of hematological disorders are described herein.

In addition, animal-based models of hematological disorders, such as those described herein, may be used to identify compounds capable of treating hematological disorders. Such animal models may be used as test substrates for the identification of drugs, pharmaceuticals, therapies, and interventions which may be effective in treating hematological disorders. For example, animal models may be exposed to a compound, suspected of exhibiting an ability to treat hematological disorders, at a sufficient concentration and for a time sufficient to elicit such an amelioration of hematological disorders in the exposed animals. The response of the animals to the exposure may be monitored by assessing the reversal of the symptoms of hematological disorders before and after treatment.

With regard to intervention, any treatments which reverse any aspect of hematological disorders should be considered as candidates for human hematological disorders therapeutic intervention. Dosages of test agents may be determined by deriving dose-response curves.

Additionally, gene expression patterns may be utilized to assess the ability of a compound to at least one symptom of hematological disorders. For example, the expression pattern of one or more genes may form part of a “gene expression profile” or “transcriptional profile” which may be then be used in such an assessment. “Gene expression profile” or “transcriptional profile”, as used herein, includes the pattern of mRNA expression obtained for a given tissue or cell type under a given set of conditions. Gene expression profiles may be generated, for example, by utilizing a differential display procedure, Northern analysis and/or RT-PCR. In one embodiment, 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 gene sequences may be used as probes and/or PCR primers for the generation and corroboration of such gene expression profiles.

Gene expression profiles may be characterized for known states, either hematological disease or normal, within the cell- and/or animal-based model systems. Subsequently, these known gene expression profiles may be compared to ascertain the effect a test compound has to modify such gene expression profiles, and to cause the profile to more closely resemble that of a more desirable profile.

For example, administration of a compound may cause the gene expression profile of a hematological disorder disease model system to more closely resemble the control system. Administration of a compound may, alternatively, cause the gene expression profile of a control system to begin to mimic hematological disorders or a hematological disorder disease state. Such a compound may, for example, be used in further characterizing the compound of interest, or may be used in the generation of additional animal models.

II. Cell- and Animal-Based Model Systems

Described herein are cell- and animal-based systems which act as models for hematological disorders. These systems may be used in a variety of applications. For example, the cell- and animal-based model systems may be used to further characterize differentially expressed genes associated with hematological disease, e.g., 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014. In addition, animal- and cell-based assays may be used as part of screening strategies designed to identify compounds which are capable of ameliorating hematological disorders, as described, below. Thus, the animal- and cell-based models may be used to identify drugs, pharmaceuticals, therapies and interventions which may be effective in treating a hematological disorder. Furthermore, such animal models may be used to determine the LD50 and the ED50 in animal subjects, and such data can be used to determine the in vivo efficacy of potential hematological disorders treatments.

A. Animal-Based Systems

Animal-based model systems of hematological disorders may include, but are not limited to, non-recombinant and engineered transgenic animals.

Non-recombinant animal models for hematological disorders may include, for example, genetic models.

Additionally, animal models exhibiting hematological disorders may be engineered by using, for example, 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 gene sequences described above, in conjunction with techniques for producing transgenic animals that are well known to those of skill in the art. For example, 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 gene sequences may be introduced into, and overexpressed in, the genome of the animal of interest, or, if endogenous 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 gene sequences are present, they may either be overexpressed or, alternatively, be disrupted in order to underexpress or inactivate 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 gene expression.

The host cells of the invention can also be used to produce non-human transgenic animals. For example, in one embodiment, a host cell of the invention is a fertilized oocyte or an embryonic stem cell into which 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014-coding sequences have been introduced. Such host cells can then be used to create non-human transgenic animals in which exogenous 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 sequences have been introduced into their genome or homologous recombinant animals in which endogenous 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 sequences have been altered. Such animals are useful for studying the function and/or activity of a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 and for identifying and/or evaluating modulators of 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, and the like. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 gene has been altered by homologous recombination between the endogenous gene and an exogenous DNA molecule introduced into a cell of the animal, e.g., an embryonic cell of the animal, prior to development of the animal.

A transgenic animal used in the methods of the invention can be created by introducing a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014-encoding nucleic acid into the male pronuclei of a fertilized oocyte, e.g., by microinjection, retroviral infection, and allowing the oocyte to develop in a pseudopregnant female foster animal. The 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 cDNA sequence can be introduced as a transgene into the genome of a non-human animal. Alternatively, a nonhuman homologue of a human 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 gene, such as a mouse or rat 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 gene, can be used as a transgene. Alternatively, a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 gene homologue, such as another 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 family member, can be isolated based on hybridization to the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 cDNA sequences and used as a transgene. Intronic sequences and polyadenylation signals can also be included in the transgene to increase the efficiency of expression of the transgene. A tissue-specific regulatory sequence(s) can be operably linked to a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 transgene to direct expression of a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein to particular cells. Methods for generating transgenic animals via embryo manipulation and microinjection, particularly animals such as mice, have become conventional in the art and are described, for example, in U.S. Pat. Nos. 4,736,866 and 4,870,009, both by Leder et al., U.S. Pat. No. 4,873,191 by Wagner et al. and in Hogan, B., Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 transgene in its genome and/or expression of 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 mRNA in tissues or cells of the animals. A transgenic founder animal can then be used to breed additional animals carrying the transgene. Moreover, transgenic animals carrying a transgene encoding a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein can further be bred to other transgenic animals carrying other transgenes.

To create a homologous recombinant animal, a vector is prepared which contains at least a portion of a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 gene into which a deletion, addition or substitution has been introduced to thereby alter, e.g., functionally disrupt, the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 gene. The 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 gene can be a human gene but more preferably, is a non-human homologue of a human 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 gene. For example, a rat 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 gene can be used to construct a homologous recombination nucleic acid molecule, e.g., a vector, suitable for altering an endogenous 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 gene in the mouse genome. In a preferred embodiment, the homologous recombination nucleic acid molecule is designed such that, upon homologous recombination, the endogenous 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 gene is functionally disrupted (i.e., no longer encodes a functional protein; also referred to as a “knock out” vector). Alternatively, the homologous recombination nucleic acid molecule can be designed such that, upon homologous recombination, the endogenous 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 gene is mutated or otherwise altered but still encodes functional protein (e.g., the upstream regulatory region can be altered to thereby alter the expression of the endogenous 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein). In the homologous recombination nucleic acid molecule, the altered portion of the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 gene is flanked at its 5′ and 3′ ends by additional nucleic acid sequence of the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 gene to allow for homologous recombination to occur between the exogenous 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 gene carried by the homologous recombination nucleic acid molecule and an endogenous 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 gene in a cell, e.g., an embryonic stem cell. The additional flanking 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 nucleic acid sequence is of sufficient length for successful homologous recombination with the endogenous gene. Typically, several kilobases of flanking DNA (both at the 5′ and 3′ ends) are included in the homologous recombination nucleic acid molecule (see, e.g., Thomas, K. R. and Capecchi, M. R. (1987) Cell 51:503 for a description of homologous recombination vectors). The homologous recombination nucleic acid molecule is introduced into a cell, e.g., an embryonic stem cell line (e.g., by electroporation) and cells in which the introduced 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 gene has homologously recombined with the endogenous 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 gene are selected (see e.g., Li, E. et al. (1992) Cell 69:915). The selected cells can then injected into a blastocyst of an animal (e.g., a mouse) to form aggregation chimeras (see e.g., Bradley, A. in Teratocarcinomas and Embryonic Stem Cells: A Practical Approach, E. J. Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination nucleic acid molecules, e.g., vectors, or homologous recombinant animals are described further in Bradley, A. (1991) Current Opinion in Biotechnology 2:823-829 and in PCT International Publication Nos.: WO 90/11354 by Le Mouellec et al.; WO 91/01140 by Smithies et al.; WO 92/0968 by Zijlstra et al.; and WO 93/04169 by Berns et al.

In another embodiment, transgenic non-human animals for use in the methods of the invention can be produced which contain selected systems which allow for regulated expression of the transgene. One example of such a system is the cre/loxP recombinase system of bacteriophage P1. For a description of the cre/loxP recombinase system, see, e.g., Lakso et al. (1992) Proc. Natl. Acad. Sci. USA 89:6232-6236. Another example of a recombinase system is the FLP recombinase system of Saccharomyces cerevisiae (O'Gorman et al. (1991) Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, I. et al. (1997) Nature 385:810-813 and PCT International Publication Nos. WO 97/07668 and WO 97/07669. In brief, a cell, e.g., a somatic cell, from the transgenic animal can be isolated and induced to exit the growth cycle and enter Gₒ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell, e.g., the somatic cell, is isolated.

The 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 transgenic animals that express 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 mRNA or a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 peptide (detected immunocytochemically, using antibodies directed against 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 epitopes) at easily detectable levels should then be further evaluated to identify those animals which display characteristic hematological disorders.

B. Cell-Based Systems

Cells that contain and express 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 gene sequences which encode a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein, and, further, exhibit cellular phenotypes associated with e.g. hematopoeisis, may be used to identify compounds that exhibit an effect. Such cells may include non-recombinant monocyte cell lines, such as U937 (ATCC# CRL-1593), THP-1 (ATCC#TIB-202), and P388D1 (ATCC# TIB-63); endothelial cells such as human umbilical vein endothelial cells (HUVECs), human microvascular endothelial cells (HMVEC), and bovine aortic endothelial cells (BAECs); as well as generic mammalian cell lines such as HeLa cells and COS cells, e.g., COS-7 (ATCC# CRL-1651), cells described supra which constitute those cells relevant to hematology. Further, such cells may include recombinant, transgenic cell lines. For example, the hematological disorders animal models of the invention, discussed above, may be used to generate cell lines, containing one or more cell types involved in e.g. hematopoeisis, that can be used as cell culture models for this disorder. While primary cultures derived from the hematological disorders model transgenic animals of the invention may be utilized, the generation of continuous cell lines is preferred. For examples of techniques which may be used to derive a continuous cell line from the transgenic animals, see Small et al., (1985) Mol. Cell Biol. 5:642-648.

Alternatively, cells of a cell type known to be involved in e.g. hematopoeisis may be transfected with sequences capable of increasing or decreasing the amount of 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 gene expression within the cell. For example, 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 gene sequences may be introduced into, and overexpressed in, the genome of the cell of interest, or, if endogenous 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 gene sequences are present, they may be either overexpressed or, alternatively disrupted in order to underexpress or inactivate 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 gene expression.

In order to overexpress a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 gene, the coding portion of the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 gene may be ligated to a regulatory sequence which is capable of driving gene expression in the cell type of interest, e.g., an endothelial cell. Such regulatory regions will be well known to those of skill in the art, and may be utilized in the absence of undue experimentation. Recombinant methods for expressing target genes are described above.

For underexpression of an endogenous 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 gene sequence, such a sequence may be isolated and engineered such that when reintroduced into the genome of the cell type of interest, the endogenous 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 alleles will be inactivated. Preferably, the engineered 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 sequence is introduced via gene targeting such that the endogenous 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 sequence is disrupted upon integration of the engineered 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 sequence into the cell's genome. Transfection of host cells with 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 genes is discussed, above.

Cells treated with compounds or transfected with 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 genes can be examined for phenotypes associated with e.g. hematopoeisis.

Transfection of 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 nucleic acid may be accomplished by using standard techniques (described in, for example, Ausubel (1989) supra). Transfected cells should be evaluated for the presence of the recombinant 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 gene sequences, for expression and accumulation of 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 mRNA, and for the presence of recombinant 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein production. In instances wherein a decrease in 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 gene expression is desired, standard techniques may be used to demonstrate whether a decrease in endogenous 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 gene expression and/or in 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein production is achieved.

Also provided are cells or a purified preparation thereof, e.g., human cells, in which an endogenous 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 is under the control of a regulatory sequence that does not normally control the expression of the endogenous 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 gene. The expression characteristics of an endogenous gene within a cell, e.g., a cell line or microorganism, can be modified by inserting a heterologous DNA regulatory element into the genome of the cell such that the inserted regulatory element is operably linked to the endogenous 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 gene. For example, an endogenous 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 gene, e.g., a gene which is “transcriptionally silent,” e.g., not normally expressed, or expressed only at very low levels, may be activated by inserting a regulatory element which is capable of promoting the expression of a normally expressed gene product in that cell. Techniques such as targeted homologous recombinations, can be used to insert the heterologous DNA as described in, e.g., Chappel, U.S. Pat. No. 5,272,071; WO 91/06667, published on May 16, 1991.

III. Predictive Medicine:

The present invention also pertains to the field of predictive medicine in which diagnostic assays, prognostic assays, and monitoring clinical trials are used for prognostic (predictive) purposes to thereby treat an individual prophylactically. Accordingly, one aspect of the present invention relates to diagnostic assays for determining 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein and/or nucleic acid expression as well as 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 activity, in the context of a biological sample (e.g., blood, serum, cells, e.g., endothelial cells, or tissue, e.g., vascular tissue) to thereby determine whether an individual is afflicted with a predisposition or is experiencing hematological disorders. The invention also provides for prognostic (or predictive) assays for determining whether an individual is at risk of developing a hematological disorder. For example, mutations in a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 gene can be assayed for in a biological sample. Such assays can be used for prognostic or predictive purpose to thereby phophylactically treat an individual prior to the onset of a hematological disorder.

Another aspect of the invention pertains to monitoring the influence of 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 modulators (e.g., anti-9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 antibodies or 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 ribozymes) on the expression or activity of 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 in clinical trials.

These and other agents are described in further detail in the following sections.

A. Diagnostic Assays

To determine whether a subject is afflicted with a disease, a biological sample may be obtained from a subject and the biological sample may be contacted with a compound or an agent capable of detecting a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein or nucleic acid (e.g., mRNA or genomic DNA) that encodes a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein, in the biological sample. A preferred agent for detecting 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 mRNA or genomic DNA is a labeled nucleic acid probe capable of hybridizing to 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 mRNA or genomic DNA. The nucleic acid probe can be, for example, the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 nucleic acid set forth in SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77 or 79, or a portion thereof, such as an oligonucleotide of at least 15, 20, 25, 30, 25, 40, 45, 50, 100, 250 or 500 nucleotides in length and sufficient to specifically hybridize under stringent conditions to 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 mRNA or genomic DNA. Other suitable probes for use in the diagnostic assays of the invention are described herein.

A preferred agent for detecting 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein in a sample is an antibody capable of binding to 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein, preferably an antibody with a detectable label. Antibodies can be polyclonal, or more preferably, monoclonal. An intact antibody, or a fragment thereof (e.g., Fab or F(ab′)2) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

The term “biological sample” is intended to include tissues, cells, and biological fluids isolated from a subject, as well as tissues, cells, and fluids present within a subject. That is, the detection method of the invention can be used to detect 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 mRNA, protein, or genomic DNA in a biological sample in vitro as well as in vivo. For example, in vitro techniques for detection of 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 mRNA include Northern hybridizations and in situ hybridizations. In vitro techniques for detection of 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein include enzyme linked immunosorbent assays (ELISAs), Western blots, immunoprecipitations and immunofluorescence. In vitro techniques for detection of 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 genomic DNA include Southern hybridizations. Furthermore, in vivo techniques for detection of 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein include introducing into a subject a labeled anti-9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 antibody. For example, the antibody can be labeled with a radioactive marker whose presence and location in a subject can be detected by standard imaging techniques.

In another embodiment, the methods further involve obtaining a control biological sample from a control subject, contacting the control sample with a compound or agent capable of detecting 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein, mRNA, or genomic DNA, such that the presence of 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein, mRNA or genomic DNA is detected in the biological sample, and comparing the presence of 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein, mRNA or genomic DNA in the control sample with the presence of 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein, mRNA or genomic DNA in the test sample.

B. Prognostic Assays

The present invention further pertains to methods for identifying subjects having or at risk of developing a disease associated with aberrant 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 expression or activity.

As used herein, the term “aberrant” includes a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 expression or activity which deviates from the wild type 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 expression or activity. Aberrant expression or activity includes increased or decreased expression or activity, as well as expression or activity which does not follow the wild type developmental pattern of expression or the subcellular pattern of expression. For example, aberrant 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 expression or activity is intended to include the cases in which a mutation in the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 gene causes the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 gene to be under-expressed or over-expressed and situations in which such mutations result in a non-functional 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein or a protein which does not function in a wild-type fashion, e.g., a protein which does not interact with a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 substrate, or one which interacts with a non-9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 substrate.

The assays described herein, such as the preceding diagnostic assays or the following assays, can be used to identify a subject having or at risk of developing a disease. A biological sample may be obtained from a subject and tested for the presence or absence of a genetic alteration. For example, such genetic alterations can be detected by ascertaining the existence of at least one of 1) a deletion of one or more nucleotides from a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 gene, 2) an addition of one or more nucleotides to a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 gene, 3) a substitution of one or more nucleotides of a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 gene, 4) a chromosomal rearrangement of a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 gene, 5) an alteration in the level of a messenger RNA transcript of a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 gene, 6) aberrant modification of a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 gene, such as of the methylation pattern of the genomic DNA, 7) the presence of a non-wild type splicing pattern of a messenger RNA transcript of a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 gene, 8) a non-wild type level of a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014-protein, 9) allelic loss of a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 gene, and 10) inappropriate post-translational modification of a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014-protein.

As described herein, there are a large number of assays known in the art which can be used for detecting genetic alterations in a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 gene. For example, a genetic alteration in a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 gene may be detected using a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 gene (see Abravaya et al. (1995) Nucleic Acids Res. 23:675-682). This method includes collecting a biological sample from a subject, isolating nucleic acid (e.g., genomic DNA, mRNA or both) from the sample, contacting the nucleic acid sample with one or more primers which specifically hybridize to a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 gene under conditions such that hybridization and amplification of the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli, J. C. et al. (1990) Proc. Natl. Acad. Sci. USA 87:1874-1878), transcriptional amplification system (Kwoh, D. Y. et al. (1989) Proc. Natl. Acad. Sci. USA 86:1173-1177), Q-Beta Replicase (Lizardi, P. M. et al. (1988) Bio-Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 gene from a biological sample can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 can be identified by hybridizing biological sample derived and control nucleic acids, e.g., DNA or RNA, to high density arrays containing hundreds or thousands of oligonucleotide probes (Cronin, M. T. et al. (1996) Human Mutation 7:244-255; Kozal, M. J. et al. (1996) Nature Medicine 2:753-759). For example, genetic mutations in 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 can be identified in two dimensional arrays containing light-generated DNA probes as described in Cronin, M. T. et al. (1996) supra. Briefly, a first hybridization array of probes can be used to scan through long stretches of DNA in a sample and control to identify base changes between the sequences by making linear arrays of sequential, overlapping probes. This step allows for the identification of point mutations. This step is followed by a second hybridization array that allows for the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 gene in a biological sample and detect mutations by comparing the sequence of the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 in the biological sample with the corresponding wild-type (control) sequence. Examples of sequencing reactions include those based on techniques developed by Maxam and Gilbert (1977) Proc. Natl. Acad. Sci. USA 74:560) or Sanger (1977) Proc. Natl. Acad. Sci. USA 74:5463). It is also contemplated that any of a variety of automated sequencing procedures can be utilized when performing the diagnostic assays (Naeve, C. W. (1995) Biotechniques 19:448-53), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes formed by hybridizing (labeled) RNA or DNA containing the wild-type 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 sequence with potentially mutant RNA or DNA obtained from a tissue sample. The double-stranded duplexes are treated with an agent which cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al. (1988) Proc. Natl. Acad Sci USA 85:4397 and Saleeba et al. (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 sequence, e.g., a wild-type 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 sequence, is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify mutations in 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 genes. For example, single strand conformation polymorphism (SSCP) may be used to detect differences in electrophoretic mobility between mutant and wild type nucleic acids (Orita et al. (1989) Proc Natl. Acad. Sci USA: 86:2766; see also Cotton (1993) Mutat. Res. 285:125-144 and Hayashi (1992) Genet. Anal. Tech. Appl. 9:73-79). Single-stranded DNA fragments of sample and control 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet 7:5).

In yet another embodiment the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to ensure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys Chem 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163); Saiki et al. (1989) Proc. Natl. Acad. Sci USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

Furthermore, the prognostic assays described herein can be used to determine whether a subject can be administered a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 modulator (e.g., an agonist, antagonist, peptidomimetic, protein, peptide, nucleic acid, or small molecule) to effectively treat a disease.

C. Monitoring of Effects During Clinical Trials

The present invention further provides methods for determining the effectiveness of a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 modulator (e.g., a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 modulator identified herein) in treating a disease. For example, the effectiveness of a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 modulator in increasing 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 gene expression, protein levels, or in upregulating 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 activity, can be monitored in clinical trials of subjects exhibiting decreased 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 gene expression, protein levels, or down-regulated 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 activity. Alternatively, the effectiveness of a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 modulator in decreasing 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 gene expression, protein levels, or in downregulating 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 activity, can be monitored in clinical trials of subjects exhibiting increased 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 gene expression, protein levels, or 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 activity. In such clinical trials, the expression or activity of a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 gene, and preferably, other genes that have been implicated in e.g. hematopoeisis or thrombosis can be used as a “read out” or marker of the phenotype of a particular cell.

For example, and not by way of limitation, genes, including 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014, that are modulated in cells by treatment with an agent which modulates 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 activity (e.g., identified in a screening assay as described herein) can be identified. Thus, to study the effect of agents which modulate 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 activity on subjects suffering from a hematological disorder in, for example, a clinical trial, cells can be isolated and RNA prepared and analyzed for the levels of expression of 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 and other genes implicated in the hematological disorders disorder. The levels of gene expression (e.g., a gene expression pattern) can be quantified by Northern blot analysis or RT-PCR, as described herein, or alternatively by measuring the amount of protein produced, by one of the methods described herein, or by measuring the levels of activity of 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 or other genes. In this way, the gene expression pattern can serve as a marker, indicative of the physiological response of the cells to the agent which modulates 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 activity. This response state may be determined before, and at various points during treatment of the individual with the agent which modulates 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 activity.

In a preferred embodiment, the present invention provides a method for monitoring the effectiveness of treatment of a subject with an agent which modulates 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 activity (e.g., an agonist, antagonist, peptidomimetic, protein, peptide, nucleic acid, or small molecule identified by the screening assays described herein) including the steps of (i) obtaining a pre-administration sample from a subject prior to administration of the agent; (ii) detecting the level of expression of a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein, mRNA, or genomic DNA in the pre-administration sample; (iii) obtaining one or more post-administration samples from the subject; (iv) detecting the level of expression or activity of the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein, mRNA, or genomic DNA in the post-administration samples; (v) comparing the level of expression or activity of the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein, mRNA, or genomic DNA in the pre-administration sample with the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein, mRNA, or genomic DNA in the post administration sample or samples; and (vi) altering the administration of the agent to the subject accordingly. For example, increased administration of the agent may be desirable to increase the expression or activity of 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 to higher levels than detected, i.e., to increase the effectiveness of the agent. Alternatively, decreased administration of the agent may be desirable to decrease expression or activity of 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 to lower levels than detected, i.e. to decrease the effectiveness of the agent. According to such an embodiment, 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 expression or activity may be used as an indicator of the effectiveness of an agent, even in the absence of an observable phenotypic response.

IV. Methods of Treatment:

The present invention provides for both prophylactic and therapeutic methods of treating a subject, e.g., a human, at risk of (or susceptible to) a disease. With regard to both prophylactic and therapeutic methods of treatment, such treatments may be specifically tailored or modified, based on knowledge obtained from the field of pharmacogenomics. “Pharmacogenomics,” as used herein, refers to the application of genomics technologies such as gene sequencing, statistical genetics, and gene expression analysis to drugs in clinical development and on the market. More specifically, the term refers to the study of how a patient's genes determine his or her response to a drug (e.g., a patient's “drug response phenotype”, or “drug response genotype”).

Thus, another aspect of the invention provides methods for tailoring an subject's prophylactic or therapeutic treatment with either the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 molecules of the present invention or 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

A. Prophylactic Methods

In one aspect, the invention provides a method for preventing in a subject, a disease by administering to the subject an agent which modulates 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 expression or 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 activity. Subjects at risk for a hematological disorder, can be identified by, for example, any or a combination of the diagnostic or prognostic assays described herein. Administration of a prophylactic agent can occur prior to the manifestation of symptoms characteristic of aberrant 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 expression or activity, such that a disease is prevented or, alternatively, delayed in its progression. Depending on the type of 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 aberrancy, for example, a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 agonist or 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 antagonist agent can be used for treating the subject. The appropriate agent can be determined based on screening assays described herein.

B. Therapeutic Methods

Described herein are methods and compositions whereby hematological disorders may be ameliorated. Certain hematological disorders disorders are brought about, at least in part, by an excessive level of a gene product, or by the presence of a gene product exhibiting an abnormal or excessive activity. As such, the reduction in the level and/or activity of such gene products would bring about the amelioration of hematological disorders. Techniques for the reduction of gene expression levels or the activity of a protein are discussed below.

Alternatively, certain other hematological disorders disorders are brought about, at least in part, by the absence or reduction of the level of gene expression, or a reduction in the level of a protein's activity. As such, an increase in the level of gene expression and/or the activity of such proteins would bring about the amelioration of hematological disorders.

In some cases, the up-regulation of a gene in a disease state reflects a protective role for that gene product in responding to the disease condition. Enhancement of such a gene's expression, or the activity of the gene product, will reinforce the protective effect it exerts. Some hematological disorders states may result from an abnormally low level of activity of such a protective gene. In these cases also, an increase in the level of gene expression and/or the activity of such gene products would bring about the amelioration of hematological disorders. Techniques for increasing target gene expression levels or target gene product activity levels are discussed herein.

Accordingly, another aspect of the invention pertains to methods of modulating 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 expression or activity for therapeutic purposes. Accordingly, in an exemplary embodiment, the modulatory method of the invention involves contacting a cell with a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 or agent that modulates one or more of the activities of 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein activity associated with the cell (e.g., an endothelial cell or an ovarian cell). An agent that modulates 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein activity can be an agent as described herein, such as a nucleic acid or a protein, a naturally-occurring target molecule of a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein (e.g., a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 ligand or substrate), a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 antibody, a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 agonist or antagonist, a peptidomimetic of a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 agonist or antagonist, or other small molecule. In one embodiment, the agent stimulates one or more 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 activities. Examples of such stimulatory agents include active 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein and a nucleic acid molecule encoding 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 that has been introduced into the cell. In another embodiment, the agent inhibits one or more 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 activities. Examples of such inhibitory agents include antisense 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 nucleic acid molecules, anti-9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 antibodies, and 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 inhibitors. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant or unwanted expression or activity of a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein or nucleic acid molecule. In one embodiment, the method involves administering an agent (e.g., an agent identified by a screening assay described herein), or combination of agents that modulates (e.g., upregulates or downregulates) 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 expression or activity. In another embodiment, the method involves administering a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein or nucleic acid molecule as therapy to compensate for reduced, aberrant, or unwanted 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 expression or activity.

Stimulation of 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 activity is desirable in situations in which 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 is abnormally downregulated and/or in which increased 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 activity is likely to have a beneficial effect. Likewise, inhibition of 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 activity is desirable in situations in which 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 is abnormally upregulated and/or in which decreased 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 activity is likely to have a beneficial effect.

### (i) Methods for Inhibiting Target Gene Expression, Synthesis, or Activity

As discussed above, genes involved in hematological disorders may cause such disorders via an increased level of gene activity. In some cases, such up-regulation may have a causative or exacerbating effect on the disease state. A variety of techniques may be used to inhibit the expression, synthesis, or activity of such genes and/or proteins.

For example, compounds such as those identified through assays described above, which exhibit inhibitory activity, may be used in accordance with the invention to at least one symptom of hematological disorders. Such molecules may include, but are not limited to, small organic molecules, peptides, antibodies, and the like.

For example, compounds can be administered that compete with endogenous ligand for the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein. The resulting reduction in the amount of ligand-bound 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein will modulate endothelial cell physiology. Compounds that can be particularly useful for this purpose include, for example, soluble proteins or peptides, such as peptides comprising one or more of the extracellular domains, or portions and/or analogs thereof, of the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein, including, for example, soluble fusion proteins such as Ig-tailed fusion proteins. (For a discussion of the production of Ig-tailed fusion proteins, see, for example, U.S. Pat. No. 5,116,964). Alternatively, compounds, such as ligand analogs or antibodies, that bind to the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 receptor site, but do not activate the protein, (e.g., receptor-ligand antagonists) can be effective in inhibiting 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein activity.

Further, antisense and ribozyme molecules which inhibit expression of the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 gene may also be used in accordance with the invention to inhibit aberrant 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 gene activity. Still further, triple helix molecules may be utilized in inhibiting aberrant 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 gene activity.

The antisense nucleic acid molecules used in the methods of the invention are typically administered to a subject or generated in situ such that they hybridize with or bind to cellular mRNA and/or genomic DNA encoding a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention include direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, an antisense nucleic acid molecule used in the methods of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid used in the methods of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haselhoff and Gerlach (1988) Nature 334:585-591)) can be used to catalytically cleave 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 mRNA transcripts to thereby inhibit translation of 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 mRNA. A ribozyme having specificity for a 577, 20739 or 57145-encoding nucleic acid can be designed based upon the nucleotide sequence of a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 cDNA disclosed herein (i.e., SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77 or 79). For example, a derivative of a Tetrahymena L-19 UVS RNA can be constructed in which the nucleotide sequence of the active site is complementary to the nucleotide sequence to be cleaved in a 577, 20739 or 57145-encoding mRNA (see, for example, Cech et al. U.S. Pat. No. 4,987,071; and Cech et al. U.S. Pat. No. 5,116,742). Alternatively, 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 mRNA can be used to select a catalytic RNA having a specific ribonuclease activity from a pool of RNA molecules (see, for example, Bartel, D. and Szostak, J. W. (1993) Science 261:1411-1418).

9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 gene expression can also be inhibited by targeting nucleotide sequences complementary to the regulatory region of the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 (e.g., the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 promoter and/or enhancers) to form triple helical structures that prevent transcription of the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 gene in target cells (see, for example, Helene, C. (1991) Anticancer Drug Des. 6(6):569-84; Helene, C. et al. (1992) Ann. N.Y. Acad. Sci. 660:27-36; and Maher, L. J. (1992) Bioassays 14(12):807-15).

Antibodies that are both specific for the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein and interfere with its activity may also be used to modulate or inhibit 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein function. Such antibodies may be generated using standard techniques described herein, against the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein itself or against peptides corresponding to portions of the protein. Such antibodies include but are not limited to polyclonal, monoclonal, Fab fragments, single chain antibodies, or chimeric antibodies.

In instances where the target gene protein is intracellular and whole antibodies are used, internalizing antibodies may be preferred. Lipofectin liposomes may be used to deliver the antibody or a fragment of the Fab region which binds to the target epitope into cells. Where fragments of the antibody are used, the smallest inhibitory fragment which binds to the target protein's binding domain is preferred. For example, peptides having an amino acid sequence corresponding to the domain of the variable region of the antibody that binds to the target gene protein may be used. Such peptides may be synthesized chemically or produced via recombinant DNA technology using methods well known in the art (described in, for example, Creighton (1983), supra; and Sambrook et al. (1989) supra). Single chain neutralizing antibodies which bind to intracellular target gene epitopes may also be administered. Such single chain antibodies may be administered, for example, by expressing nucleotide sequences encoding single-chain antibodies within the target cell population by utilizing, for example, techniques such as those described in Marasco et al. (1993) Proc. Natl. Acad. Sci. USA 90:7889-7893).

In some instances, the target gene protein is extracellular, or is a transmembrane protein, such as the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein. Antibodies that are specific for one or more extracellular domains of the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein, for example, and that interfere with its activity, are particularly useful in treating hematological disorders or a hematological disorder. Such antibodies are especially efficient because they can access the target domains directly from the bloodstream. Any of the administration techniques described below which are appropriate for peptide administration may be utilized to effectively administer inhibitory target gene antibodies to their site of action.

### (ii) Methods for Restoring or Enhancing Target Gene Activity

Genes that cause hematological disorders may be underexpressed within disease situations. Alternatively, the activity of the protein products of such genes may be decreased, leading to the development of hematological disorders. Such down-regulation of gene expression or decrease of protein activity might have a causative or exacerbating effect on the disease state.

In some cases, genes that are up-regulated in the disease state might be exerting a protective effect. A variety of techniques may be used to increase the expression, synthesis, or activity of genes and/or proteins that exert a protective effect in response to hematological disorders conditions.

Described in this section are methods whereby the level 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 activity may be increased to levels wherein hematological disorders are ameliorated. The level of 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 activity may be increased, for example, by either increasing the level of 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 gene expression or by increasing the level of active 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein which is present.

For example, a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein, at a level sufficient to at least one symptom of hematological disorders may be administered to a patient exhibiting such symptoms. Any of the techniques discussed below may be used for such administration. One of skill in the art will readily know how to determine the concentration of effective, non-toxic doses of the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein, utilizing techniques such as those described below.

Additionally, RNA sequences encoding a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein may be directly administered to a patient exhibiting hematological disorders, at a concentration sufficient to produce a level of 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein such that hematological disorders are ameliorated. Any of the techniques discussed below, which achieve intracellular administration of compounds, such as, for example, liposome administration, may be used for the administration of such RNA molecules. The RNA molecules may be produced, for example, by recombinant techniques such as those described herein.

Further, subjects may be treated by gene replacement therapy. One or more copies of a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 gene, or a portion thereof, that directs the production of a normal 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein with 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 function, may be inserted into cells using vectors which include, but are not limited to adenovirus, adeno-associated virus, and retrovirus vectors, in addition to other particles that introduce DNA into cells, such as liposomes. Additionally, techniques such as those described above may be used for the introduction of 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 gene sequences into human cells.

Cells, preferably, autologous cells, containing 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 expressing gene sequences may then be introduced or reintroduced into the subject at positions which allow for the amelioration of hematological disorders. Such cell replacement techniques may be preferred, for example, when the gene product is a secreted, extracellular gene product.

C. Pharmaceutical Compositions

Another aspect of the invention pertains to methods for treating a subject suffering from a disease. These methods involve administering to a subject an agent which modulates 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 expression or activity (e.g., an agent identified by a screening assay described herein), or a combination of such agents. In another embodiment, the method involves administering to a subject a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein or nucleic acid molecule as therapy to compensate for reduced, aberrant, or unwanted 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 expression or activity.

Stimulation of 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 activity is desirable in situations in which 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 is abnormally downregulated and/or in which increased 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 activity is likely to have a beneficial effect. Likewise, inhibition of 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 activity is desirable in situations in which 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 is abnormally upregulated and/or in which decreased 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 activity is likely to have a beneficial effect.

The agents which modulate 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 activity can be administered to a subject using pharmaceutical compositions suitable for such administration. Such compositions typically comprise the agent (e.g., nucleic acid molecule, protein, or antibody) and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition used in the therapeutic methods of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the agent that modulates 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 activity (e.g., a fragment of a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein or an anti-9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The agents that modulate 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 activity can also be prepared in the form of suppositories (e.g., with conventional suppository bases such as cocoa butter and other glycerides) or retention enemas for rectal delivery.

In one embodiment, the agents that modulate 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 activity are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the agent that modulates 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 activity and the particular therapeutic effect to be achieved, and the limitations inherent in the art of compounding such an agent for the treatment of subjects.

Toxicity and therapeutic efficacy of such agents can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and can be expressed as the ratio LD50/ED50. Agents which exhibit large therapeutic indices are preferred. While agents that exhibit toxic side effects may be used, care should be taken to design a delivery system that targets such agents to the site of affected tissue in order to minimize potential damage to uninfected cells and, thereby, reduce side effects.

The data obtained from the cell culture assays and animal studies can be used in formulating a range of dosage for use in humans. The dosage of such 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 modulating agents lies preferably within a range of circulating concentrations that include the ED50 with little or no toxicity. The dosage may vary within this range depending upon the dosage form employed and the route of administration utilized. For any agent used in the therapeutic methods of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

In a preferred example, a subject is treated with antibody, protein, or polypeptide in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody, protein, or polypeptide used for treatment may increase or decrease over the course of a particular treatment. Changes in dosage may result and become apparent from the results of diagnostic assays as described herein.

The present invention encompasses agents which modulate expression or activity. An agent may, for example, be a small molecule. For example, such small molecules include, but are not limited to, peptides, peptidomimetics, amino acids, amino acid analogs, polynucleotides, polynucleotide analogs, nucleotides, nucleotide analogs, organic or inorganic compounds (i.e, including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. It is understood that appropriate doses of small molecule agents depends upon a number of factors within the ken of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention.

Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram). It is furthermore understood that appropriate doses of a small molecule depend upon the potency of the small molecule with respect to the expression or activity to be modulated. Such appropriate doses may be determined using the assays described herein. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

Further, an antibody (or fragment thereof) may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates of the invention can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, alpha-interferon, beta-interferon, nerve growth factor, platelet derived growth factor, tissue plasminogen activator; or biological response modifiers such as, for example, lymphokines, interleukin-1 (“IL-1”), interleukin-2 (“IL-2”), interleukin-6 (“IL-6”), granulocyte macrophase colony stimulating factor (“GM-CSF”), granulocyte colony stimulating factor (“G-CSF”), or other growth factors.

Techniques for conjugating such therapeutic moiety to antibodies are well known, see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982). Alternatively, an antibody can be conjugated to a second antibody to form an antibody heteroconjugate as described by Segal in U.S. Pat. No. 4,676,980.

The nucleic acid molecules used in the methods of the invention can be inserted into vectors and used as gene therapy vectors. Gene therapy vectors can be delivered to a subject by, for example, intravenous injection, local administration (see U.S. Pat. No. 5,328,470) or by stereotactic injection (see, e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

D. Pharmacogenomics

In conjunction with the therapeutic methods of the invention, pharmacogenomics (i.e., the study of the relationship between a subject's genotype and that subject's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer an agent which modulates 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 activity, as well as tailoring the dosage and/or therapeutic regimen of treatment with an agent which modulates 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 activity.

Pharmacogenomics deals with clinically significant hereditary variations in the response to drugs due to altered drug disposition and abnormal action in affected persons. See, for example, Eichelbaum, M. et al. (1996) Clin. Exp. Pharmacol. Physiol. 23(10-11): 983-985 and Linder, M. W. et al. (1997) Clin. Chem. 43(2):254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate aminopeptidase deficiency (G6PD) is a common inherited enzymopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants). Such a high-resolution genetic map can be compared to a map of the genome of each of a statistically significant number of patients taking part in a Phase II/III drug trial to identify markers associated with a particular observed drug response or side effect. Alternatively, such a high resolution map can be generated from a combination of some ten million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach” can be utilized to identify genes that predict drug response. According to this method, if a gene that encodes a drug target is known (e.g., a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein used in the methods of the present invention), all common variants of that gene can be fairly easily identified in the population and it can be determined if having one version of the gene versus another is associated with a particular drug response.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and the cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Alternatively, a method termed the “gene expression profiling” can be utilized to identify genes that predict drug response. For example, the gene expression of an animal dosed with a drug (e.g., a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 molecule or 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 modulator used in the methods of the present invention) can give an indication whether gene pathways related to toxicity have been turned on.

Information generated from more than one of the above pharmacogenomics approaches can be used to determine appropriate dosage and treatment regimens for prophylactic or therapeutic treatment of a subject. This knowledge, when applied to dosing or drug selection, can avoid adverse reactions or therapeutic failure and, thus, enhance therapeutic or prophylactic efficiency when treating a subject suffering from a hematological disease.

V. Recombinant Expression Vectors and Host Cells Used in the Methods of the Invention

The methods of the invention (e.g., the screening assays described herein) include the use of vectors, preferably expression vectors, containing a nucleic acid encoding a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors to be used in the methods of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel (1990) Methods Enzymol. 185:3-7. Regulatory sequences include those which direct constitutive expression of a nucleotide sequence in many types of host cells and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein (e.g., 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 proteins, mutant forms of 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 proteins, fusion proteins, and the like).

The recombinant expression vectors to be used in the methods of the invention can be designed for expression of 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 proteins in prokaryotic or eukaryotic cells. For example, 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 proteins can be expressed in bacterial cells such as E. coli, insect cells (using baculovirus expression vectors), yeast cells, or mammalian cells. Suitable host cells are discussed further in Goeddel (1990) supra. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified fusion proteins can be utilized in 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 activity assays, (e.g., direct assays or competitive assays described in detail below), or to generate antibodies specific for 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 proteins. In a preferred embodiment, a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 fusion protein expressed in a retroviral expression vector of the present invention can be utilized to infect bone marrow cells which are subsequently transplanted into irradiated recipients. The pathology of the subject recipient is then examined after sufficient time has passed (e.g., six weeks).

In another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J. et al., Molecular Cloning: A Laboratory Manual. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid).

The methods of the invention may further use a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific, or cell type specific expression of antisense RNA. The antisense expression vector can be in the form of a recombinant plasmid, phagemid, or attenuated virus in which antisense nucleic acids are produced under the control of a high efficiency regulatory region, the activity of which can be determined by the cell type into which the vector is introduced. For a discussion of the regulation of gene expression using antisense genes, see Weintraub, H. et al., Antisense RNA as a molecular tool for genetic analysis, Reviews—Trends in Genetics, Vol. 1(1) 1986.

Another aspect of the invention pertains to the use of host cells into which a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 nucleic acid molecule of the invention is introduced, e.g., a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 nucleic acid molecule within a recombinant expression vector or a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 nucleic acid molecule containing sequences which allow it to homologously recombine into a specific site of the host cell's genome. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

A host cell used in the methods of the invention, such as a prokaryotic or eukaryotic host cell in culture, can be used to produce (i.e., express) a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein. Accordingly, the invention further provides methods for producing a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein using the host cells of the invention. In one embodiment, the method comprises culturing the host cell of the invention (into which a recombinant expression vector encoding a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein has been introduced) in a suitable medium such that a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein is produced. In another embodiment, the method further comprises isolating a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein from the medium or the host cell.

VI. Isolated Nucleic Acid Molecules Used in the Methods of the Invention

The methods of the invention include the use of isolated nucleic acid molecules that encode 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 proteins or biologically active portions thereof, as well as nucleic acid fragments sufficient for use as hybridization probes to identify 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014-encoding nucleic acid molecules (e.g., 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 mRNA) and fragments for use as PCR primers for the amplification or mutation of 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

A nucleic acid molecule used in the methods of the present invention, e.g., a nucleic acid molecule having the nucleotide sequence of SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77 or 79, or a portion thereof, can be isolated using standard molecular biology techniques and the sequence information provided herein. Using all or portion of the nucleic acid sequence of SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77 or 79, as a hybridization probe, 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 nucleic acid molecules can be isolated using standard hybridization and cloning techniques (e.g., as described in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989).

Moreover, a nucleic acid molecule encompassing all or a portion of SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77 or 79 can be isolated by the polymerase chain reaction (PCR) using synthetic oligonucleotide primers designed based upon the sequence of SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77 or 79.

A nucleic acid used in the methods of the invention can be amplified using cDNA, mRNA or, alternatively, genomic DNA as a template and appropriate oligonucleotide primers according to standard PCR amplification techniques. Furthermore, oligonucleotides corresponding to 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 nucleotide sequences can be prepared by standard synthetic techniques, e.g., using an automated DNA synthesizer.

In a preferred embodiment, the isolated nucleic acid molecules used in the methods of the invention comprise the nucleotide sequence shown in SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77 or 79, a complement of the nucleotide sequence shown in SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77 or 79, or a portion of any of these nucleotide sequences. A nucleic acid molecule which is complementary to the nucleotide sequence shown in SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77 or 79, is one which is sufficiently complementary to the nucleotide sequence shown in SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77 or 79 such that it can hybridize to the nucleotide sequence shown in SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77 or 79 thereby forming a stable duplex.

In still another preferred embodiment, an isolated nucleic acid molecule used in the methods of the present invention comprises a nucleotide sequence which is at least about 55%, 60%, 65%, 70%, 75%, 80%, 85%, 90%, 95%, 96%, 97%, 98%, 99% or more identical to the entire length of the nucleotide sequence shown in SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77 or 79, or a portion of any of this nucleotide sequence.

Moreover, the nucleic acid molecules used in the methods of the invention can comprise only a portion of the nucleic acid sequence of SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77 or 79, for example, a fragment which can be used as a probe or primer or a fragment encoding a portion of a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein, e.g., a biologically active portion of a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein. The probe/primer typically comprises substantially purified oligonucleotide. The oligonucleotide typically comprises a region of nucleotide sequence that hybridizes under stringent conditions to at least about 12 or 15, preferably about 20 or 25, more preferably about 30, 35, 40, 45, 50, 55, 60, 65, or 75 consecutive nucleotides of a sense sequence of SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77 or 79, of an anti-sense sequence of SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77 or 79, or of a naturally occurring allelic variant or mutant of SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77 or 79. In one embodiment, a nucleic acid molecule used in the methods of the present invention comprises a nucleotide sequence which is greater than 100, 100-200, 200-300, 300-400, 400-500, 500-600, 600-700, 700-800, 800-900, 900-1000, 1000-1100, 1100-1200, 1200-1300, or more nucleotides in length and hybridizes under stringent hybridization conditions to a nucleic acid molecule of SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77 or 79.

As used herein, the term “hybridizes under stringent conditions” is intended to describe conditions for hybridization and washing under which nucleotide sequences that are significantly identical or homologous to each other remain hybridized to each other. Preferably, the conditions are such that sequences at least about 70%, more preferably at least about 80%, even more preferably at least about 85% or 90% identical to each other remain hybridized to each other. Such stringent conditions are known to those skilled in the art and can be found in Current Protocols in Molecular Biology, Ausubel et al., eds., John Wiley & Sons, Inc. (1995), sections 2, 4 and 6. Additional stringent conditions can be found in Molecular Cloning: A Laboratory Manual, Sambrook et al., Cold Spring Harbor Press, Cold Spring Harbor, N.Y. (1989), chapters 7, 9 and 11. A preferred, non-limiting example of stringent hybridization conditions includes hybridization in 4× sodium chloride/sodium citrate (SSC), at about 65-70° C. (or hybridization in 4×SSC plus 50% formamide at about 42-50° C.) followed by one or more washes in 1×SSC, at about 65-70° C. A preferred, non-limiting example of highly stringent hybridization conditions includes hybridization in 1×SSC, at about 65-70° C. (or hybridization in 1×SSC plus 50% formamide at about 42-50° C.) followed by one or more washes in 0.3×SSC, at about 65-70° C. A preferred, non-limiting example of reduced stringency hybridization conditions includes hybridization in 4×SSC, at about 50-60° C. (or alternatively hybridization in 6×SSC plus 50% formamide at about 40-45° C.) followed by one or more washes in 2×SSC, at about 50-60° C. Ranges intermediate to the above-recited values, e.g., at 65-70° C. or at 42-50° C. are also intended to be encompassed by the present invention. SSPE (1×SSPE is 0.15M NaCl, 10 mM NaH₂PO₄, and 1.25 mM EDTA, pH 7.4) can be substituted for SSC (1×SSC is 0.15M NaCl and 15 mM sodium citrate) in the hybridization and wash buffers; washes are performed for 15 minutes each after hybridization is complete. The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.)=2(# of A+T bases)+4(# of G+C bases). For hybrids between 18 and 49 base pairs in length, Tₘ(° C.)=81.5+16.6(log₁₀[Na⁺])+0.41(% G+C)−(600/N), where N is the number of bases in the hybrid, and [Na⁺] is the concentration of sodium ions in the hybridization buffer ([Na⁺] for 1×SSC=0.165 M). It will also be recognized by the skilled practitioner that additional reagents may be added to hybridization and/or wash buffers to decrease non-specific hybridization of nucleic acid molecules to membranes, for example, nitrocellulose or nylon membranes, including but not limited to blocking agents (e.g., BSA or salmon or herring sperm carrier DNA), detergents (e.g., SDS), chelating agents (e.g., EDTA), Ficoll, PVP and the like. When using nylon membranes, in particular, an additional preferred, non-limiting example of stringent hybridization conditions is hybridization in 0.25-0.5M NaH₂PO₄, 7% SDS at about 65° C., followed by one or more washes at 0.02M NaH₂PO₄, 1% SDS at 65° C., see e.g., Church and Gilbert (1984) Proc. Natl. Acad. Sci. USA 81:1991-1995, (or alternatively 0.2×SSC, 1% SDS).

In preferred embodiments, the probe further comprises a label group attached thereto, e.g., the label group can be a radioisotope, a fluorescent compound, an enzyme, or an enzyme co-factor. Such probes can be used as a part of a diagnostic test kit for identifying cells or tissue which misexpress a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein, such as by measuring a level of a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014-encoding nucleic acid in a sample of cells from a subject e.g., detecting 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 mRNA levels or determining whether a genomic 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 gene has been mutated or deleted.

The methods of the invention further encompass the use of nucleic acid molecules that differ from the nucleotide sequence shown in SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77 or 79, due to degeneracy of the genetic code and thus encode the same 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 proteins as those encoded by the nucleotide sequence shown in SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77 or 79. In another embodiment, an isolated nucleic acid molecule included in the methods of the invention has a nucleotide sequence encoding a protein having an amino acid sequence shown in SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78 or 80.

The methods of the invention further include the use of allelic variants of human 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014, e.g., functional and non-functional allelic variants. Functional allelic variants are naturally occurring amino acid sequence variants of the human 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein that maintain a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 activity. Functional allelic variants will typically contain only conservative substitution of one or more amino acids of SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78 or 80, or substitution, deletion or insertion of non-critical residues in non-critical regions of the protein.

Non-functional allelic variants are naturally occurring amino acid sequence variants of the human 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein that do not have a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 activity. Non-functional allelic variants will typically contain a non-conservative substitution, deletion, or insertion or premature truncation of the amino acid sequence of SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78 or 80, or a substitution, insertion or deletion in critical residues or critical regions of the protein.

The methods of the present invention may further use non-human orthologues of the human 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein. Orthologues of the human 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein are proteins that are isolated from non-human organisms and possess the same 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 activity.

The methods of the present invention further include the use of nucleic acid molecules comprising the nucleotide sequence of SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77 or 79, or a portion thereof, in which a mutation has been introduced. The mutation may lead to amino acid substitutions at “non-essential” amino acid residues or at “essential” amino acid residues. A “non-essential” amino acid residue is a residue that can be altered from the wild-type sequence of 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 (e.g., the sequence of SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78 or 80) without altering the biological activity, whereas an “essential” amino acid residue is required for biological activity. For example, amino acid residues that are conserved among the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 proteins of the present invention are not likely to be amenable to alteration.

Mutations can be introduced into SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77 or 79, by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein is preferably replaced with another amino acid residue from the same side chain family. Alternatively, in another embodiment, mutations can be introduced randomly along all or part of a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 coding sequence, such as by saturation mutagenesis, and the resultant mutants can be screened for 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 biological activity to identify mutants that retain activity. Following mutagenesis of SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77 or 79, the encoded protein can be expressed recombinantly and the activity of the protein can be determined using the assay described herein.

Another aspect of the invention pertains to the use of isolated nucleic acid molecules which are antisense to the nucleotide sequence of SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77 or 79. An “antisense” nucleic acid comprises a nucleotide sequence which is complementary to a “sense” nucleic acid encoding a protein, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 coding strand, or to only a portion thereof. In one embodiment, an antisense nucleic acid molecule is antisense to a “coding region” of the coding strand of a nucleotide sequence encoding a 148, 302 or 567. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014. The term “noncoding region” refers to 5′ and 3′ sequences which flank the coding region that are not translated into amino acids (also referred to as 5′ and 3′ untranslated regions).

Given the coding strand sequences encoding 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 disclosed herein, antisense nucleic acids of the invention can be designed according to the rules of Watson and Crick base pairing. The antisense nucleic acid molecule can be complementary to the entire coding region of 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 mRNA, but more preferably is an oligonucleotide which is antisense to only a portion of the coding or noncoding region of 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 mRNA. For example, the antisense oligonucleotide can be complementary to the region surrounding the translation start site of 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 mRNA. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest). Antisense nucleic acid molecules used in the methods of the invention are further described above, in section IV.

In yet another embodiment, the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 nucleic acid molecules used in the methods of the present invention can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acids (see Hyrup B. et al. (1996) Bioorganic & Medicinal Chemistry 4 (1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup B. et al. (1996) supra; Perry-O'Keefe et al. (1996) Proc. Natl. Acad. Sci. 93:14670-675.

PNAs of 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 nucleic acid molecules can be used in the therapeutic and diagnostic applications described herein. For example, PNAs can be used as antisense or antigene agents for sequence-specific modulation of gene expression by, for example, inducing transcription or translation arrest or inhibiting replication. PNAs of 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 nucleic acid molecules can also be used in the analysis of single base pair mutations in a gene, (e.g., by PNA-directed PCR clamping); as ‘artificial restriction enzymes’ when used in combination with other enzymes, (e.g., S1 nucleases (Hyrup B. et al. (1996) supra)); or as probes or primers for DNA sequencing or hybridization (Hyrup B. et al. (1996) supra; Perry-O'Keefe et al. (1996) supra).

In another embodiment, PNAs of 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 can be modified, (e.g., to enhance their stability or cellular uptake), by attaching lipophilic or other helper groups to PNA, by the formation of PNA-DNA chimeras, or by the use of liposomes or other techniques of drug delivery known in the art. For example, PNA-DNA chimeras of 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 nucleic acid molecules can be generated which may combine the advantageous properties of PNA and DNA. Such chimeras allow DNA recognition enzymes, (e.g., RNAse H and DNA polymerases), to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup B. et al. (1996) supra). The synthesis of PNA-DNA chimeras can be performed as described in Hyrup B. et al. (1996) supra and Finn P. J. et al. (1996) Nucleic Acids Res. 24 (17): 3357-63. For example, a DNA chain can be synthesized on a solid support using standard phosphoramidite coupling chemistry and modified nucleoside analogs, e.g., 5′-(4-methoxytrityl)amino-5′-deoxy-thymidine phosphoramidite, can be used as a between the PNA and the 5′ end of DNA (Mag, M. et al. (1989) Nucleic Acid Res. 17: 5973-88). PNA monomers are then coupled in a stepwise manner to produce a chimeric molecule with a 5′ PNA segment and a 3′ DNA segment (Finn P. J. et al. (1996) supra). Alternatively, chimeric molecules can be synthesized with a 5′ DNA segment and a 3′ PNA segment (Peterser, K. H. et al. (1975) Bioorganic Med. Chem. Lett. 5: 1119-11124).

In other embodiments, the oligonucleotide used in the methods of the invention may include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. WO88/09810) or the blood-brain barrier (see, e.g., PCT Publication No. WO89/10134). In addition, oligonucleotides can be modified with hybridization-triggered cleavage agents (See, e.g., Krol et al. (1988) Bio-Techniques 6:958-976) or intercalating agents. (See, e.g., Zon (1988) Pharm. Res. 5:539-549). To this end, the oligonucleotide may be conjugated to another molecule, (e.g., a peptide, hybridization triggered cross-linking agent, transport agent, or hybridization-triggered cleavage agent).

VII. Isolated 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 proteins and Anti-9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 Antibodies Used in the Methods of the Invention

The methods of the invention include the use of isolated 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 proteins, and biologically active portions thereof, as well as polypeptide fragments suitable for use as immunogens to raise anti-9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 antibodies. In one embodiment, native 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 proteins can be isolated from cells or tissue sources by an appropriate purification scheme using standard protein purification techniques. In another embodiment, 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 proteins are produced by recombinant DNA techniques. Alternative to recombinant expression, a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein or polypeptide can be synthesized chemically using standard peptide synthesis techniques.

As used herein, a “biologically active portion” of a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein includes a fragment of a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein having a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 activity. Biologically active portions of a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein include peptides comprising amino acid sequences sufficiently identical to or derived from the amino acid sequence of the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein, e.g., the amino acid sequence shown in SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78 or 80, which include fewer amino acids than the full length 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 proteins, and exhibit at least one activity of a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein. Typically, biologically active portions comprise a domain or motif with at least one activity of the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein (e.g., the N-terminal region of the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein that is believed to be involved in the regulation of apoptotic activity). A biologically active portion of a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein can be a polypeptide which is, for example, 25, 50, 75, 100, 125, 150, 175, 200, 250, 300 or more amino acids in length. Biologically active portions of a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein can be used as targets for developing agents which modulate a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 activity.

In a preferred embodiment, the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein used in the methods of the invention has an amino acid sequence shown in SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78 or 80. In other embodiments, the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein is substantially identical to SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78 or 80, and retains the functional activity of the protein of SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78 or 80, yet differs in amino acid sequence due to natural allelic variation or mutagenesis, as described in detail in subsection V above. Accordingly, in another embodiment, the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein used in the methods of the invention is a protein which comprises an amino acid sequence at least about 50%, 55%, 60%, 65%, 70%, 75%, 80%, 85%, 90%, 95%, 96%, 97%, 98%, 99% or more identical to SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78 or 80.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-identical sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence (e.g., when aligning a second sequence to the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 amino acid sequence of SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78 or 80 having 500 amino acid residues, at least 75, preferably at least 150, more preferably at least 225, even more preferably at least 300, and even more preferably at least 400 or more amino acid residues are aligned). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (J. Mol. Biol. 48:444-453 (1970)) algorithm which has been incorporated into the GAP program in the GCG software package using either a Blosum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package using a NWSgapdna.CMP matrix and a gap weight of 40, 50, 60, 70, or 80 and a length weight of 1, 2, 3, 4, 5, or 6. In another embodiment, the percent identity between two amino acid or nucleotide sequences is determined using the algorithm of E. Meyers and W. Miller (Comput. Appl. Biosci. 4:11-17 (1988)) which has been incorporated into the ALIGN program (version 2.0 or 2.0U), using a PAM 120 weight residue table, a gap length penalty of 12 and a gap penalty of 4.

The methods of the invention may also use 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 chimeric or fusion proteins. As used herein, a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 “chimeric protein” or “fusion protein” comprises a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 polypeptide operatively linked to a non-9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 polypeptide. An “9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 polypeptide” refers to a polypeptide having an amino acid sequence corresponding to a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 molecule, whereas a “non-9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 polypeptide” refers to a polypeptide having an amino acid sequence corresponding to a protein which is not substantially homologous to the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein, e.g., a protein which is different from the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein and which is derived from the same or a different organism. Within a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 fusion protein the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 polypeptide can correspond to all or a portion of a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein. In a preferred embodiment, a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 fusion protein comprises at least one biologically active portion of a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein. In another preferred embodiment, a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 fusion protein comprises at least two biologically active portions of a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein. Within the fusion protein, the term “operatively linked” is intended to indicate that the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 polypeptide and the non-9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 polypeptide are fused in-frame to each other. The non-9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 polypeptide can be fused to the N-terminus or C-terminus of the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 polypeptide.

For example, in one embodiment, the fusion protein is a GST-9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 fusion protein in which the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 sequences are fused to the C-terminus of the GST sequences. Such fusion proteins can facilitate the purification of recombinant 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014.

In another embodiment, this fusion protein is a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein containing a heterologous signal sequence at its N-terminus. In certain host cells (e.g., mammalian host cells), expression and/or secretion of 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 can be increased through use of a heterologous signal sequence.

The 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 fusion proteins used in the methods of the invention can be incorporated into pharmaceutical compositions and administered to a subject in vivo. The 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 fusion proteins can be used to affect the bioavailability of a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 substrate. Use of 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 fusion proteins may be useful therapeutically for the treatment of disorders caused by, for example, (i) aberrant modification or mutation of a gene encoding a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein; (ii) mis-regulation of the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 gene; and (iii) aberrant post-translational modification of a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein.

Moreover, the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014-fusion proteins used in the methods of the invention can be used as immunogens to produce anti-9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 antibodies in a subject, to purify 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 ligands and in screening assays to identify molecules which inhibit the interaction of 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 with a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 substrate.

Preferably, a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 chimeric or fusion protein used in the methods of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypeptide). A 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014-encoding nucleic acid can be cloned into such an expression vector such that the fusion moiety is linked in-frame to the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein.

The present invention also pertains to the use of variants of the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 proteins which function as either 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 agonists (mimetics) or as 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 antagonists. Variants of the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 proteins can be generated by mutagenesis, e.g., discrete point mutation or truncation of a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein. An agonist of the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 proteins can retain substantially the same, or a subset, of the biological activities of the naturally occurring form of a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein. An antagonist of a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein can inhibit one or more of the activities of the naturally occurring form of the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein by, for example, competitively modulating a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014-mediated activity of a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein.

In one embodiment, variants of a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein which function as either 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 agonists (mimetics) or as 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 antagonists can be identified by screening combinatorial libraries of mutants, e.g., truncation mutants, of a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein for 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein agonist or antagonist activity. In one embodiment, a variegated library of 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 variants is generated by combinatorial mutagenesis at the nucleic acid level and is encoded by a variegated gene library. A variegated library of 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 variants can be produced by, for example, enzymatically ligating a mixture of synthetic oligonucleotides into gene sequences such that a degenerate set of potential 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 sequences is expressible as individual polypeptides, or alternatively, as a set of larger fusion proteins (e.g., for phage display) containing the set of 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 sequences therein. There are a variety of methods which can be used to produce libraries of potential 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477).

In addition, libraries of fragments of a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein coding sequence can be used to generate a variegated population of 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 fragments for screening and subsequent selection of variants of a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein. In one embodiment, a library of coding sequence fragments can be generated by treating a double stranded PCR fragment of a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 proteins. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique which enhances the frequency of functional mutants in the libraries, can be used in combination with the screening assays to identify 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 variants (Arkin and Yourvan (1992) Proc. Natl. Acad. Sci. USA 89:7811-7815; Delgrave et al. (1993) Protein Engineering 6(3):327-331).

The methods of the present invention further include the use of anti-9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 antibodies. An isolated 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein, or a portion or fragment thereof, can be used as an immunogen to generate antibodies that bind 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 using standard techniques for polyclonal and monoclonal antibody preparation. A full-length 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein can be used or, alternatively, antigenic peptide fragments of 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 can be used as immunogens. The antigenic peptide of 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 comprises at least 8 amino acid residues of the amino acid sequence shown in SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78 or 80 and encompasses an epitope of 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 such that an antibody raised against the peptide forms a specific immune complex with the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein. Preferably, the antigenic peptide comprises at least 10 amino acid residues, more preferably at least 15 amino acid residues, even more preferably at least 20 amino acid residues, and most preferably at least 30 amino acid residues.

Preferred epitopes encompassed by the antigenic peptide are regions of 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 that are located on the surface of the protein, e.g., hydrophilic regions, as well as regions with high antigenicity.

A 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 immunogen is typically used to prepare antibodies by immunizing a suitable subject, (e.g., rabbit, goat, mouse, or other mammal) with the immunogen. An appropriate immunogenic preparation can contain, for example, recombinantly expressed 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein or a chemically synthesized 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 polypeptide. The preparation can further include an adjuvant, such as Freund's complete or incomplete adjuvant, or similar immunostimulatory agent. Immunization of a suitable subject with an immunogenic 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 preparation induces a polyclonal anti-9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 antibody response.

The term “antibody” as used herein refers to immunoglobulin molecules and immunologically active portions of immunoglobulin molecules, i.e., molecules that contain an antigen binding site which specifically binds (immunoreacts with) an antigen, such as a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 molecules. The term “monoclonal antibody” or “monoclonal antibody composition”, as used herein, refers to a population of antibody molecules that contain only one species of an antigen binding site capable of immunoreacting with a particular epitope of 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014. A monoclonal antibody composition thus typically displays a single binding affinity for a particular 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein with which it immunoreacts.

Polyclonal anti-9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 antibodies can be prepared as described above by immunizing a suitable subject with a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 immunogen. The anti-9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 antibody titer in the immunized subject can be monitored over time by standard techniques, such as with an enzyme linked immunosorbent assay (ELISA) using immobilized 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014. If desired, the antibody molecules directed against 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 can be isolated from the mammal (e.g., from the blood) and further purified by well known techniques, such as protein A chromatography to obtain the IgG fraction. At an appropriate time after immunization, e.g., when the anti-9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 antibody titers are highest, antibody-producing cells can be obtained from the subject and used to prepare monoclonal antibodies by standard techniques, such as the hybridoma technique originally described by Kohler and Milstein (1975) Nature 256:495-497) (see also, Brown et al. (1981) J. Immunol. 127:539-46; Brown et al. (1980) J. Biol. Chem. 255:4980-83; Yeh et al. (1976) Proc. Natl. Acad. Sci. USA 76:2927-31; and Yeh et al. (1982) Int. J. Cancer 29:269-75), the more recent human B cell hybridoma technique (Kozbor et al. (1983) Immunol Today 4:72), the EBV-hybridoma technique (Cole et al. (1985) Monoclonal Antibodies and Cancer Therapy, Alan R. Liss, Inc., pp. 77-96) or trioma techniques. The technology for producing monoclonal antibody hybridomas is well known (see generally Kenneth, R. H. in Monoclonal Antibodies: A New Dimension In Biological Analyses, Plenum Publishing Corp., New York, N.Y. (1980); Lerner, E. A. (1981) Yale J. Biol. Med. 54:387-402; Gefter, M. L. et al. (1977) Somatic Cell Genet. 3:231-36). Briefly, an immortal cell line (typically a myeloma) is fused to lymphocytes (typically splenocytes) from a mammal immunized with a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 immunogen as described above, and the culture supernatants of the resulting hybridoma cells are screened to identify a hybridoma producing a monoclonal antibody that binds 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014.

Any of the many well known protocols used for fusing lymphocytes and immortalized cell lines can be applied for the purpose of generating an anti-9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 monoclonal antibody (see, e.g., G. Galfre et al. (1977) Nature 266:55052; Gefter et al. (1977) supra; Lerner (1981) supra; and Kenneth (1980) supra). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014, e.g., using a standard ELISA assay.

Alternative to preparing monoclonal antibody-secreting hybridomas, a monoclonal anti-9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 antibody can be identified and isolated by screening a recombinant combinatorial immunoglobulin library (e.g., an antibody phage display library) with 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 to thereby isolate immunoglobulin library members that bind 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, Ladner et al. U.S. Pat. No. 5,223,409; Kang et al. PCT International Publication No. WO 92/18619; Dower et al. PCT International Publication No. WO 91/17271; Winter et al. PCT International Publication WO 92/20791; Markland et al. PCT International Publication No. WO 92/15679; Breitling et al. PCT International Publication WO 93/01288; McCafferty et al. PCT International Publication No. WO 92/01047; Garrard et al. PCT International Publication No. WO 92/09690; Ladner et al. PCT International Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734; Hawkins et al. (1992) J. Mol. Biol. 226:889-896; Clarkson et al. (1991) Nature 352:624-628; Gram et al. (1992) Proc. Natl. Acad. Sci. USA 89:3576-3580; Garrad et al. (1991) Bio/Technology 9:1373-1377; Hoogenboom et al. (1991) Nuc. Acid Res. 19:4133-4137; Barbas et al. (1991) Proc. Natl. Acad. Sci. USA 88:7978-7982; and McCafferty et al. (1990) Nature 348:552-554.

Additionally, recombinant anti-9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 antibodies, such as chimeric and humanized monoclonal antibodies, comprising both human and non-human portions, which can be made using standard recombinant DNA techniques, are within the scope of the methods of the invention. Such chimeric and humanized monoclonal antibodies can be produced by recombinant DNA techniques known in the art, for example using methods described in Robinson et al. International Application No. PCT/US86/02269; Akira, et al. European Patent Application 184,187; Taniguchi, M., European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Canc. Res. 47:999-1005; Wood et al. (1985) Nature 314:446-449; Shaw et al. (1988) J. Natl. Cancer Inst. 80:1553-1559; Morrison, S. L. (1985) Science 229:1202-1207; Oi et al. (1986) BioTechniques 4:214; Winter U.S. Pat. No. 5,225,539; Jones et al. (1986) Nature 321:552-525; Verhoeyan et al. (1988) Science 239:1534; and Beidler et al. (1988) J. Immunol. 141:4053-4060.

An anti-9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 antibody can be used to detect 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein (e.g., in a cellular lysate or cell supernatant) in order to evaluate the abundance and pattern of expression of the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 protein. Anti-9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, □-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

This invention is further illustrated by the following examples which should not be construed as limiting. The contents of all references, patents and published patent applications cited throughout this application, as well as the Figure and the Sequence Listing is incorporated herein by reference.

### EXAMPLES

### Example 1

### Tissue Distribution of Using TaqMan™ Analysis

This example describes the TaqMan™ procedure. The Taqman™ procedure is a quantitative, reverse transcription PCR-based approach for detecting mRNA. The RT-PCR reaction exploits the 5′ nuclease activity of AmpliTaq Gold™ DNA Polymerase to cleave a TaqMan™ probe during PCR. Briefly, cDNA was generated from the samples of interest, e.g., heart, kidney, liver, skeletal muscle, and various vessels, and used as the starting material for PCR amplification. In addition to the 5′ and 3′ gene-specific primers, a gene-specific oligonucleotide probe (complementary to the region being amplified) was included in the reaction (i.e., the Taqman™ probe). The TaqMan™ probe includes the oligonucleotide with a fluorescent reporter dye covalently linked to the 5′ end of the probe (such as FAM (6-carboxyfluorescein), TET (6-carboxy-4,7,2′,7′-tetrachlorofluorescein), JOE (6-carboxy-4,5-dichloro-2,7-dimethoxyfluorescein), or VIC) and a quencher dye (TAMRA (6-carboxy-N,N,N′,N′-tetramethylrhodamine) at the 3′ end of the probe.

During the PCR reaction, cleavage of the probe separates the reporter dye and the quencher dye, resulting in increased fluorescence of the reporter. Accumulation of PCR products is detected directly by monitoring the increase in fluorescence of the reporter dye. When the probe is intact, the proximity of the reporter dye to the quencher dye results in suppression of the reporter fluorescence. During PCR, if the target of interest is present, the probe specifically anneals between the forward and reverse primer sites. The 5′-3′ nucleolytic activity of the AmpliTaq™ Gold DNA Polymerase cleaves the probe between the reporter and the quencher only if the probe hybridizes to the target. The probe fragments are then displaced from the target, and polymerization of the strand continues. The 3′ end of the probe is blocked to prevent extension of the probe during PCR. This process occurs in every cycle and does not interfere with the exponential accumulation of product. RNA was prepared using the trizol method and treated with DNase to remove contaminating genomic DNA. cDNA was synthesized using standard techniques. Mock cDNA synthesis in the absence of reverse transcriptase resulted in samples with no detectable PCR amplification of the control gene confirms efficient removal of genomic DNA contamination.

Equivalents

Those skilled in the art will recognize, or be able to ascertain using no more than routine experimentation, many equivalents to the specific embodiments of the invention described herein. Such equivalents are intended to be encompassed by the following claims.

1-22. (canceled)

23. A method for identifying a compound capable of treating a hematological disorder, comprising:
a) combining a compound to be tested with a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 polypeptide under conditions suitable for binding of the test compound to the polypeptide or under conditions suitable for modulation of the activity of the polypeptide; and
b) detecting binding of the test compound to the polypeptide to thereby identify a compound which binds to the polypeptide, or determining the effect of the test compound on the activity of the polypeptide to thereby identify a compound which modulates the activity of the polypeptide, thereby identifying a compound capable of treating a hematological disorder.

24. The method of claim 23, wherein the compound is selected from the group consisting of a small molecule, a peptide or an antibody.

25. The method of claim 23, wherein the polypeptide further comprises heterologous sequences.

26. The method of claim 23, wherein the polypeptide is an isolated polypeptide, a membrane-bound form of an isolated polypeptide or a cell comprising the polypeptide.

27. The method of claim 26, wherein the cell is a hematological cell.

28. The method of claim 23, wherein the hematological disorder is a disorder associated with, but not limited to, anemia, neutropenia or thrombocytopenia.

29. The method of claim 23, wherein the binding of the test compound to the polypeptide is detected by a method selected from the group consisting of:
a) a competition binding assay;
b) an immunoassay; and
c) a yeast two-hybrid assay.

30. A method for identifying a compound capable of treating a hematological disorder, comprising:
a) combining a compound to be tested with a host cell expressing a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 polypeptide under conditions suitable for binding of the test compound to the polypeptide or under conditions suitable for modulation of the activity of the polypeptide; and
b) detecting binding of the test compound to the polypeptide to thereby identify a compound which binds to the polypeptide, or determining the effect of the test compound on the activity of the polypeptide to thereby identify a compound which modulates the activity of the polypeptide, thereby identifying a compound capable of treating a hematological disorder.

31. The method of claim 30, wherein the compound is selected from the group consisting of a small molecule, a peptide, an antibody or an antisense nucleic acid molecule.

32. The method of claim 30, wherein the polypeptide further comprises heterologous sequences.

33. The method of claim 30, wherein the host cell is a hematological cell.

34. The method of claim 30, wherein the hematological disorder is a disorder associated with, but not limited to, anemia, neutropenia or thrombocytopenia.

35. The method of claim 30, wherein the binding of the test compound to the polypeptide is detected by a method selected from the group consisting of:
a) a competition binding assay;
b) an immunoassay; and
c) a yeast two-hybrid assay.

36. A method of identifying a subject having a hematological disorder, or at risk for developing a hematological disorder comprising:
a) contacting a sample obtained from the subject comprising polypeptides with a 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 binding substance; and
b) detecting the presence of a polypeptide in the sample that binds to the 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 binding substance, thereby identifying a subject having a hematological disorder, or at risk for developing a hematological disorder.

37. The method of claim 36, wherein the binding substance is an antibody.

38. The method of claim 36, wherein the binding substance is detectably labeled.
